(19) United States
(12) Patent Application Publication
(10) Pub. No.: US 2015/0196285 A1
(43) Pub. Date: Jul. 16, 2015

(54) CALIBRATION TOOL, IMAGING APPARATUS FOR DIAGNOSIS, AND CALIBRATION METHOD OF IMAGING APPARATUS FOR DIAGNOSIS

(71) Applicant: Terumo Kabushiki Kaisha, Tokyo (JP)

(72) Inventor: Isao MORI, Tokyo (JP)

(73) Assignee: TERUMO KABUSHIKI KAISHA, Tokyo (JP)

(21) Appl. No.: 14/668,045

(22) Filed: Mar. 25, 2015

Related U.S. Application Data
(63) Continuation of application No. PCT/JP2012/006131, filed on Sep. 26, 2012.

(51) Int. Cl. A61B 8/00; A61B 5/00; A61B 8/12

(52) U.S. Cl. CPC A61B 8/58; A61B 8/12; A61B 5/0066; A61B 8/4416; A61B 5/0084

(57) ABSTRACT

An imaging apparatus is disclosed for diagnosis, which has multiple transceivers and can perform position correction of a generated tomographic image, based on a distance difference in an axial direction and/or an angular difference in a circumferential direction between the respective transceivers. The imaging apparatus generates an ultrasound tomographic image and an optical tomographic image by using a signal obtained in such a way that a transceiver in which an ultrasound transceiver and an optical transceiver are arranged moves in the axial direction while rotating inside a lumen of a measurement object body. The apparatus generates data for the ultrasound tomographic image and data for the optical tomographic image of a calibration tool having a reflection section. The apparatus calculates the angular difference in the circumferential direction around the axis between the ultrasound transceiver and the optical transceiver, based on positional information of the reflection section.

### CROSS-REFERENCES TO RELATED APPLICATIONS

This application is a continuation of International Application No. PCT/JP2012/006131 filed on Sep. 26, 2012, the entire content of which is incorporated herein by reference.

### TECHNICAL FIELD

The disclosure generally relates to an imaging apparatus for diagnosis, a calibration tool for calibrating the imaging apparatus for diagnosis, and a calibration method of the imaging apparatus for diagnosis using the calibration tool.

### BACKGROUND DISCUSSION

An imaging apparatus for diagnosis has been used for diagnosis of arteriosclerosis, preoperative diagnosis in performing endovascular treatment using a high-performance catheter such as a balloon catheter, a stent or the like, or for confirmation of postoperative results.

The imaging apparatus for diagnosis includes an intravascular ultrasound (IVUS) diagnosis apparatus and an optical coherence tomography (OCT) diagnosis apparatus, which respectively have different characteristics.

In addition, in recent years, an imaging apparatus for diagnosis (imaging apparatus for diagnosis which includes an ultrasound transceiver capable of transmitting and receiving ultrasound waves and an optical transceiver capable of transmitting and receiving light) which has an IVUS function and an OCT function in combination has also been proposed (for example, refer to JP-A-11-56752 and JP-T-2010-508973). According to this imaging apparatus for diagnosis, single scanning can generate both a cross-sectional image (ultrasound cross-sectional image) utilizing IVUS characteristics, which can enable measurement for a deep region and a cross-sectional image (optical cross-sectional image) utilizing OCT characteristics with a high resolution measurement.

### SUMMARY

Since both an IVUS transceiver and an OCT transceiver can have a fixed size, the transmitting and receiving positions of these transceivers cannot be made completely coincident with each other. Accordingly, the IVUS transceiver and the OCT transceiver can be arranged by being deviated or offset from each other in the axial direction, or are arranged to have an angular difference in the circumferential direction so that the transmitting and receiving direction of ultrasound waves is different from the transmitting and receiving direction of light, around the axis.

When an ultrasound tomographic image and an optical tomographic image are generated, a distance difference in the axial direction and/or an angular difference in the circumferential direction between the IVUS transceiver and the OCT transceiver needs to be considered.

However, it can be difficult to accurately measure the distance difference in the axial direction and/or the angular difference in the circumferential direction between the IVUS transceiver and the OCT transceiver. In addition, there can be errors between the distance difference or the angular difference according to the specifications and the actual distance difference or the actual angular difference. Consequently, both of these are not always coincident with each other.

For this reason, in an imaging apparatus for diagnosis which has multiple transceivers, it can be desirable to adopt a configuration in which the distance difference in the axial direction and/or the angular difference in the circumferential direction between the respective transceivers are accurately calculated by using respectively generated tomographic images, and in which position correction can be performed based on the calculated result in order to align one tomographic image with the other tomographic image.

In accordance with an exemplary embodiment, an imaging apparatus for diagnosis is disclosed, which can have multiple transceivers, in which position correction of a generated tomographic image can be performed based on a distance difference in an axial direction and/or an angular difference in a circumferential direction between the respective transceivers.

In accordance with an exemplary embodiment, an imaging apparatus is disclosed for diagnosis in which when a transceiver having a first transceiver arranged to transmit and receive a first signal and a second transceiver arranged to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver. The apparatus can include generation means for generating the first tomographic image of a calibration tool based on the first signal transmitted and received by the first transceiver, and for generating the second tomographic image of the calibration tool based on the second signal transmitted and received by the second transceiver, with regard to the calibration tool which has a reflection section arranged to reflect the first signal and the second signal and has a lumen into which the transceiver is inserted, calculation means for calculating an angular difference around an axis between the first transceiver and the second transceiver, based on positional information of the reflection section which is detected on the first tomographic image of the calibration tool and positional information of the reflection section which is detected on the second tomographic image of the calibration tool, and correction means for correcting an angle around the axis of the first tomographic image or the second tomographic image inside the lumen of the measurement object body according to the angular difference calculated by the calculation means, when displaying the first tomographic image and the second tomographic image inside the lumen of the measurement object body.

In accordance with an exemplary embodiment, an imaging apparatus is disclosed for diagnosis which has multiple transceivers, which can perform position correction of a generated tomographic image, based on a distance difference in an axial direction and/or an angular difference in a circumferential direction between the respective transceivers.

In accordance with an exemplary embodiment, a calibration method is disclosed of an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, the method comprising: a generation step of generating the first tomographic image of a calibration tool based on the first signal transmitted and received by the first transceiver and generating the second tomographic image of the calibration tool based on the second signal transmitted and received by the second transceiver, with regard to the calibration tool which has a reflection section arranged to reflect the first signal and the second signal and has a lumen into which the transceiver is inserted; a calculation step of calculating an angular difference in a circumferential direction around an axis between the first transceiver and the second transceiver, based on positional information of the reflection section which is detected on the first tomographic image of the calibration tool and positional information of the reflection section which is detected on the second tomographic image of the calibration tool; and a correction step of correcting an angle in the circumferential direction of the first tomographic image or the second tomographic image inside the lumen of the measurement object body according to the angular difference calculated by the calculation step, when displaying the first tomographic image and the second tomographic image inside the lumen of the measurement object body.

In accordance with an exemplary embodiment, a non-transitory computer-readable recording medium is disclosed with a program stored therein which causes a computer to execute each process of the calibration method as disclosed herein.

In accordance with an exemplary embodiment, a calibration tool is disclosed for calibrating an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, comprising: a reflection section to reflect the first signal and the second signal, wherein the reflection section is arranged in a spiral shape along the axial direction; and a lumen into which the transceiver is inserted.

In accordance with an exemplary embodiment, a calibration tool is disclosed for calibrating an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, comprising: a reflection section to reflect the first signal and the second signal, wherein the reflection section is formed to have a straight line which is substantially parallel to the axial direction is arranged; and a lumen into which the transceiver is inserted.

Other characteristics and advantages of the disclosure will become apparent from the following description made with reference to the accompanying drawings. In the accompanying drawings, the same reference numerals are given to the same or similar configuration elements.

### BRIEF DESCRIPTION OF THE DRAWINGS

The accompanying drawings are incorporated in the description, configure a part of the description, represent embodiments of the imaging apparatus for diagnosis, the calibration tool, and the calibration method, and are used to describe principles of the imaging apparatus for diagnosis, the calibration tool, and the calibration method together with the description.

FIG. 1 is a view illustrating an external configuration of an imaging apparatus for diagnosis according to an exemplary embodiment of the present disclosure.

FIG. 2 is a view illustrating an overall configuration of a probe unit and a cross-sectional configuration of a distal end portion.

FIG. 3A is a diagram illustrating a cross-sectional configuration of an imaging core.

FIG. 3B is a cross-sectional view taken along a plane substantially orthogonal to the rotation center axis as an ultrasonic transmitting and receiving position.

FIG. 3C is a cross-sectional view taken along a plane substantially orthogonal to the rotation center axis at an optical transmitting and receiving position.

FIG. 4 is a diagram illustrating a functional configuration of the imaging apparatus for diagnosis.

FIG. 5 is a diagram illustrating a functional configuration of a signal processing unit in the imaging apparatus for diagnosis.

FIG. 6A is a view illustrating an example of a calibration tool for calibrating the imaging apparatus for diagnosis.

FIG. 6B is a view illustrating an example of a calibration tool for calibrating the imaging apparatus for diagnosis.

FIG. 6C is a view illustrating an example of a calibration tool for calibrating the imaging apparatus for diagnosis.

FIG. 7A illustrates a state where the imaging core 220 inserted into the calibration tool 600 during the calibration process is viewed from an opening side of the calibration tool 600.

FIG. 7B illustrates a configuration of the line data obtained by transmitting and receiving the ultrasound waves or the light at each rotation angle.

FIG. 8A is a diagram illustrating an example of data of an ultrasound tomographic image acquired by scanning the calibration tool.

FIG. 8B is a diagram illustrating an example of data of an optical tomographic image acquired by scanning the calibration tool.

FIG. 9 is a diagram schematically illustrating data of the ultrasound tomographic image and the optical tomographic image, which are acquired by scanning the calibration tool.

FIG. 10 is a flowchart illustrating flow in a calibration process in a calibration unit.

FIG. 11 is a graph illustrating an angular difference in a circumferential direction between an ultrasound transceiver and an optical transceiver, which is calculated by using the ultrasound tomographic image and the optical tomographic image.

FIG. 12 is a view illustrating an example of the calibration tool for calibrating the imaging apparatus for diagnosis 100.

FIG. 13 is a diagram schematically illustrating data of the ultrasound tomographic image and the optical tomographic image, which are acquired by scanning the calibration tool.

FIG. 14 is a graph illustrating the angular difference in the circumferential direction between the ultrasound transceiver and the optical transceiver, which is calculated by using the ultrasound tomographic image and the optical tomographic image.

FIG. 15 is a view illustrating an example of the calibration tool for calibrating the imaging apparatus for diagnosis 100.

FIG. 16 is a diagram schematically illustrating data of the ultrasound tomographic image and the optical tomographic image, which are acquired by scanning the calibration tool.

FIG. 17 is a graph illustrating deviation in an axial direction and in the circumferential direction between the ultrasound transceiver and the optical transceiver, which is calculated by using the ultrasound tomographic image and the optical tomographic image.

FIG. 18 is a view illustrating an example of the calibration tool for calibrating the imaging apparatus for diagnosis 100.

FIG. 19 is a graph illustrating deviation in the axial direction and in the circumferential direction between the ultrasound transceiver and the optical transceiver, which is calculated by using the ultrasound tomographic image and the optical tomographic image.

FIG. 20 is a view illustrating a positional relationship between the calibration tool and an imaging core.

### DETAILED DESCRIPTION

Hereinafter, each embodiment will be described in detail with reference to the accompanying drawings, when necessary.

### 1. External Configuration of Imaging Apparatus for Diagnosis

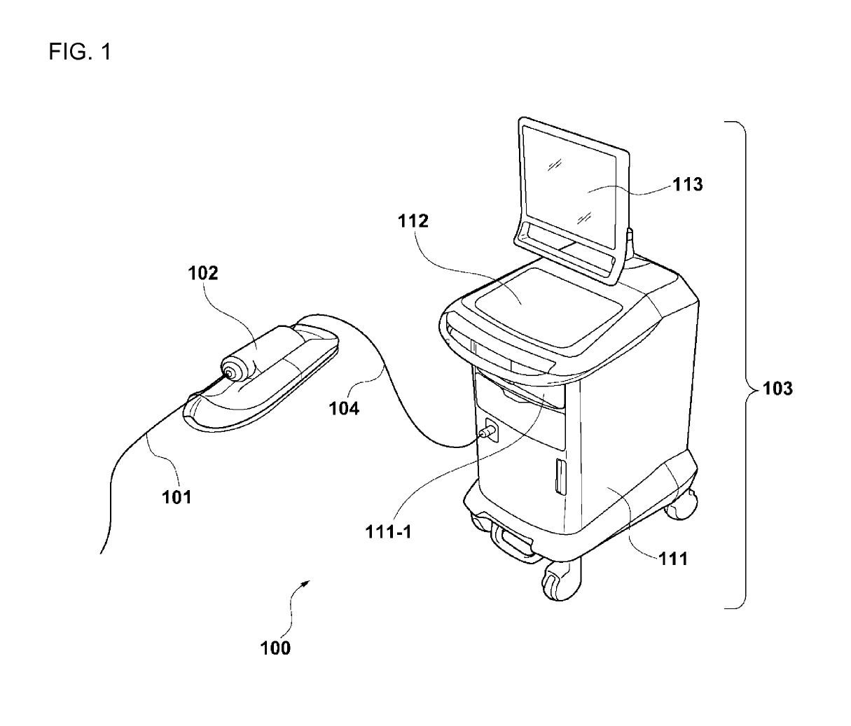

FIG. 1 is a view illustrating an external configuration of an imaging apparatus for diagnosis (imaging apparatus for diagnosis, which can include an IVUS function and an OCT function) 100 according to an exemplary embodiment of the present disclosure.

As illustrated in FIG. 1, the imaging apparatus for diagnosis 100 can include a probe unit 101, a scanner and pull-back unit 102, and an operation control device 103. The scanner and pull-back unit 102 and the operation control device 103 can be connected to each other by a signal line 104 so that various signals can be transmitted.

The probe unit 101 has an internally inserted imaging core which is directly inserted into a blood vessel (measurement object body) and can include an ultrasound transceiver which transmits ultrasound waves into the blood vessel based on a pulse signal and which receives reflected waves from the inside of the blood vessel, and an optical transceiver which continuously transmits transmitted light (measurement light) into the blood vessel and which continuously receives reflected light from the inside of the blood vessel. The imaging apparatus for diagnosis 100 measures an intravascular state by using the imaging core.

The probe unit 101 can be detachably attached to the scanner and pull-back unit 102. A motor incorporated in the scanner and pull-back unit 102 is driven, thereby regulating an intravascular operation in the axial direction and an intravascular operation in the rotation direction around the axis of the imaging core, which can be internally inserted into the probe unit 101. In addition, the scanner and pull-back unit 102 acquires the reflected wave received by the ultrasound transceiver and the reflected light received by the optical transceiver, and transmits the reflected wave and the reflected light to the operation control device 103.

The operation control device 103 can include a function for inputting various setting values upon each measurement, and a function for processing data obtained by the measurement and for displaying an intravascular tomographic image.

In the operation control device 103, the reference numeral 111 represents a main body control unit which generates ultrasound data based on the reflected waves obtained by the measurement, and which generates an ultrasound tomographic image by processing line data generated based on the ultrasound data. Furthermore, the main body control unit 111 generates interference light data by causing the reflected light obtained by the measurement to interfere with reference light obtained by separating the light from a light source, and generates an optical tomographic image by processing the generated line data based on the interference light data.

The reference numeral 111-1 represents a printer and DVD recorder, which prints a processing result in the main body control unit 111 or stores the processing result as data. The reference numeral 112 represents an operation panel, and a user inputs various setting values and instructions via the operation panel 112. The reference numeral 113 represents an LCD monitor as a display device, which displays a tomographic image generated in the main body control unit 111.

### 2. Overall Configuration of Probe Unit and Cross-Sectional Configuration of Distal End Portion

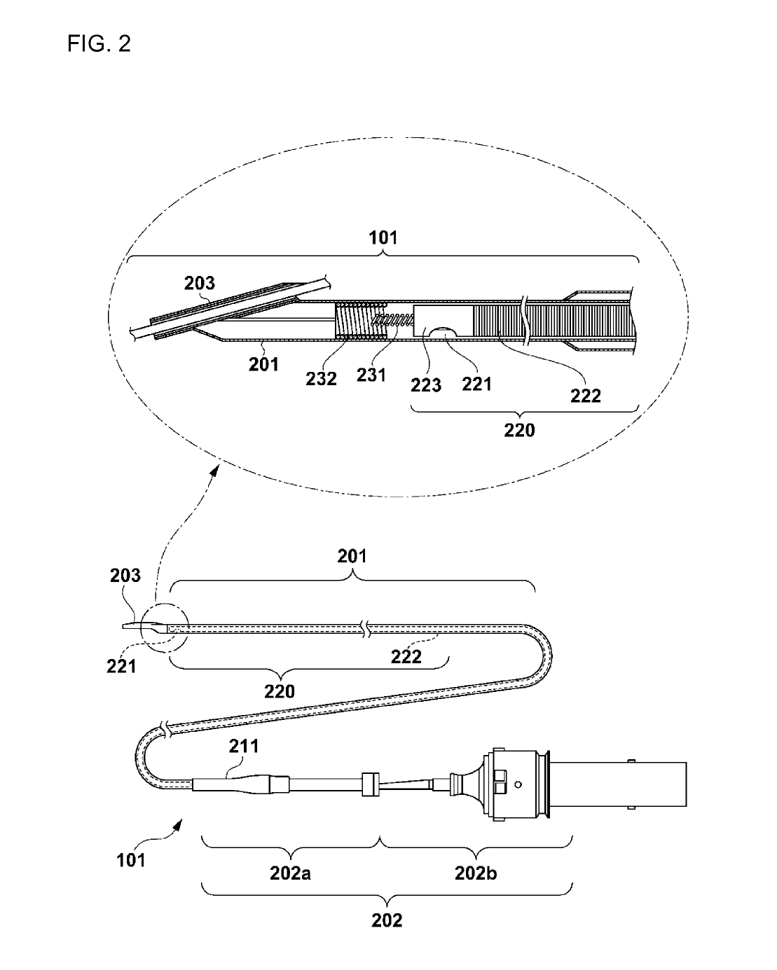

Next, an overall configuration of the probe unit 101 and a cross-sectional configuration of a distal end portion will be described with reference to FIG. 2. As illustrated in FIG. 2, the probe unit 101 is configured to include a long catheter sheath 201 to be inserted into the blood vessel and a connector unit 202 to be arranged on the front side of a user, and which can be operated by the user without being inserted into the blood vessel. The distal end of the catheter sheath 201 includes a tube possessing a guide wire lumen configured to receive a guide wire. The catheter sheath 201 has a lumen, which is continuously formed from a connection portion with the guidewire lumen tube 203 to a connection portion with the connector unit 202.

An imaging core 220 which internally can include a transceiver 221 in which the ultrasound transceiver for transmitting and receiving the ultrasound waves and the optical transceiver for transmitting and receiving the light are arranged, and which can include a coil-shaped drive shaft 222 internally including an electrical signal cable and an optical fiber cable and transmitting rotary drive power for rotating the transceiver 221 is inserted into the lumen of the catheter sheath 201 over substantially the entire length of the catheter sheath 201.

The connector unit 202 can include a sheath connector 202a configured to be integral with a proximal end of the catheter sheath 201, and a drive shaft connector 202b which is configured to rotatably fix the drive shaft 222 to the proximal end of the drive shaft 222.

An anti-kink protector 211 is disposed in a boundary section between the sheath connector 202a and the catheter sheath 201, which can help maintain a predetermined rigidity, and can help prevent bending (kinking) caused by a rapid change in physical properties.

The proximal end of the drive shaft connector 202b is detachably attached to the scanner and pull-back unit 102.

Next, the cross-sectional configuration of the distal end portion of the probe unit 101 will be described. The imaging core 220 can include a housing 223 having the transceiver 221 in which the ultrasound transceiver for transmitting and receiving the ultrasound waves and the optical transceiver for transmitting and receiving the light are arranged, and including the drive shaft 222 for transmitting the rotary drive power for rotating the housing 223, which is inserted into the lumen of the catheter sheath 201 over substantially the entire length of the catheter sheath 201, thereby forming the probe unit 101.

The drive shaft 222 can cause the transceiver 221 to perform a rotary operation and an axial operation with respect to the catheter sheath 201, and has a property, which is flexible and can transmit rotation well. For example, the drive shaft 222 can be configured to have a multiplex and multilayer contact coil or the like formed of a metal wire such as a stainless steel wire or the like. Then, an electric signal cable and an optical fiber cable (optical fiber cable in a single mode) can be arranged inside the drive shaft 222.

The housing 223 has a shape in which a short cylindrical metal pipe partially has a cutout portion, and, for example, is formed by being cut out from a metal ingot, or is molded by means of metal powder injection molding (MIM). In addition, an elastic member 231 having a short coil shape can be disposed on the distal end side of the housing 223.

The elastic member 231 is obtained by forming a stainless steel wire into a coil shape. The elastic member 231 is arranged on the distal end side, thereby help preventing the imaging core 220 from being caught on the inside of the catheter sheath 201 when the imaging core 220 is moved forward and rearward.

The reference numeral 232 represents a reinforcement coil, which is disposed in order to help prevent rapid bending of the distal end portion of the catheter, sheath 201.

The guidewire lumen tube 203 has a guidewire lumen into which a guidewire can be inserted. The guidewire lumen tube 203 is used in receiving the guidewire inserted into the blood vessel in advance and allowing the guidewire to guide the catheter sheath 201 to a lesion.

### 3. Cross-Sectional Configuration of Imaging Core

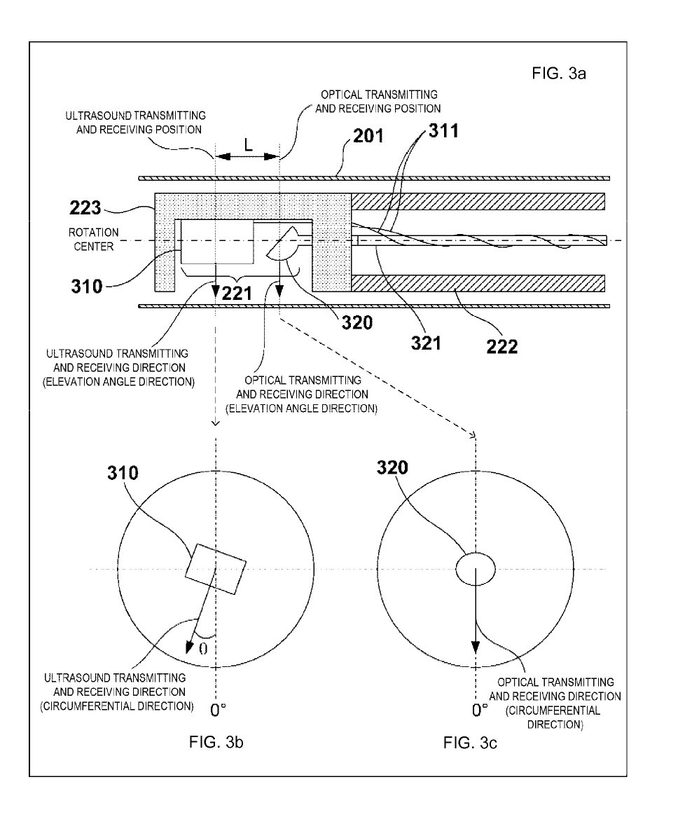

Next, a cross-sectional configuration of the imaging core 220 and an arrangement for the ultrasound transceiver and the optical transceiver will be described. FIGS. 3A-3C are diagrams illustrating the cross-sectional configuration of the imaging core, the arrangement for the ultrasound transceiver, and the optical transceiver, respectively.

As illustrated in FIG. 3A, the transceiver 221 arranged inside the housing 223 can include an ultrasound transceiver 310 and an optical transceiver 320. The ultrasound transceiver 310 and the optical transceiver 320 are respectively arranged by leaving a distance L therebetween along the axial direction on the rotation center axis (on the one-dot chain line in FIG. 3A) of the drive shaft 222.

In accordance with an exemplary embodiment, the ultrasound transceiver 310 can be arranged on the distal end side of the probe unit 101, and the optical transceiver 320 can be arranged on the proximal end side of the probe unit 101.

In addition, the ultrasound transceiver 310 and the optical transceiver 320 are attached inside the housing 223 so that an ultrasound transmitting and receiving direction (elevation angle direction) of the ultrasound transceiver 310 and a light transmitting and receiving direction (elevation angle direction) of the optical transceiver 320 are respectively, for example, approximately 90° with respect to the axial direction of the drive shaft 222. In accordance with an exemplary embodiment, the ultrasound transceiver 310 can be attached to the optical transceiver 320 by causing each transmitting and receiving direction to be slightly deviated from 90° so as not to receive the reflection on a surface inside the lumen of the catheter sheath 201.

An electric signal cable 311 connected to the ultrasound transceiver 310 and an optical fiber cable 321 connected to the optical transceiver 320 are arranged inside the drive shaft 222. The electric signal cable 311 can be wound around the optical fiber cable 321 in a spiral shape.

FIG. 3B is a cross-sectional view taken along a plane substantially orthogonal to the rotation center axis at an ultrasound transmitting and receiving position. As illustrated in FIG. 3B, when a downward direction from the paper surface is zero degrees, the ultrasound transmitting and receiving direction (circumferential direction (also referred to as an azimuth angle direction)) of the ultrasound transceiver 310 is θ degrees.

FIG. 3C is a cross-sectional view taken along a plane substantially orthogonal to the rotation center axis at an optical transmitting and receiving position. As illustrated in FIG. 3C, when the downward direction from the paper surface is zero degrees, the light transmitting and receiving direction (circumferential direction) of the optical transceiver 320 is zero degrees. That is, the ultrasound transceiver 310 and the optical transceiver 320 are arranged so that the ultrasound transmitting and receiving direction (circumferential direction) of the ultrasound transceiver 310 and the light transmitting and receiving direction (circumferential direction) of the optical transceiver 320 mutually have an angular difference of θ degrees.

### 4. Functional Configuration of Imaging Apparatus for Diagnosis

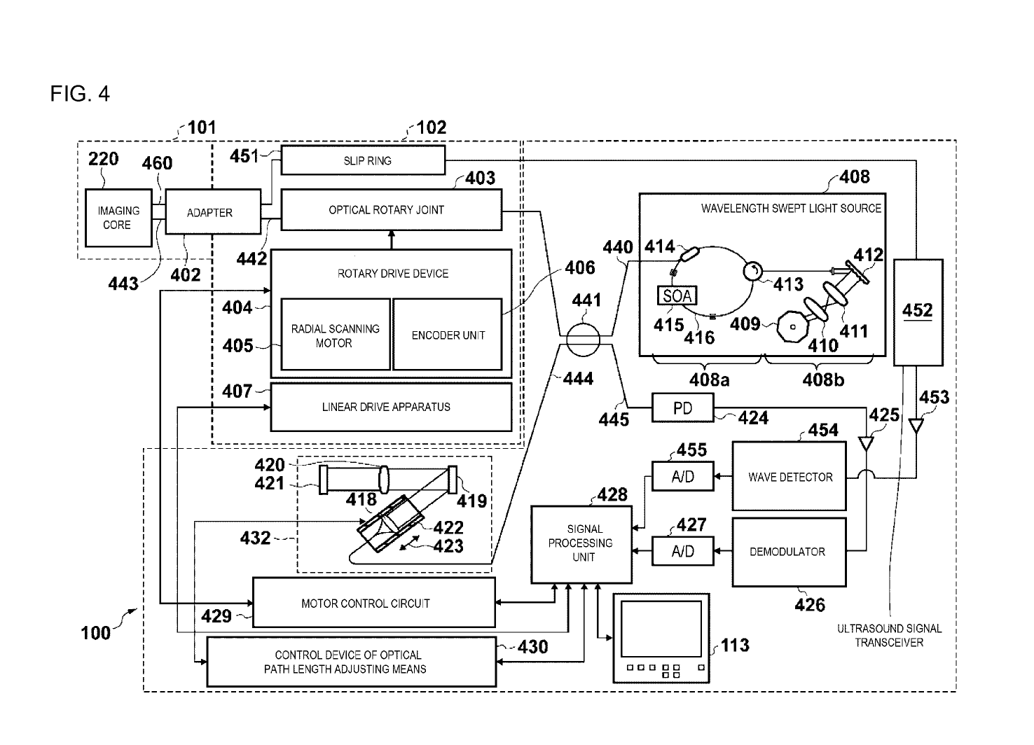

Next, a functional configuration of the imaging apparatus for diagnosis 100 will be described. FIG. 4 is a diagram illustrating the functional configuration of the imaging apparatus for diagnosis 100 which includes an IVUS function and an OCT function (here, a wavelength swept-type OCT as an example) in combination. An imaging apparatus for diagnosis including the IVUS function and other OCT functions in combination also has the same functional configuration. Therefore, description thereof will be omitted herein.
(1) IVUS Function

The imaging core 220 can include the ultrasound transceiver 310 inside the distal end of the imaging core 220. The ultrasound transceiver 310 can transmit ultrasound waves to biological tissues inside the blood vessel based on pulse waves transmitted by an ultrasound signal transceiver 452, receives reflected waves (echoes) of the ultrasonic waves, and transmits the reflected waves to the ultrasound signal transceiver 452 as an ultrasound signal via an adapter 402 and a slip ring 451.

In the scanner and pull-back unit 102, a rotary drive portion side of the slip ring 451 is rotatably driven by a radial scanning motor 405 of a rotary drive device 404. In addition, a rotation angle of the radial scanning motor 405 is detected by an encoder unit 406. Furthermore, the scanner and pull-back unit 102 can include a linear drive apparatus 407, and can regulate the axial operation of the imaging core 220 based on a signal from a signal processing unit 428.

The ultrasound signal transceiver 452 can include a transmitting wave circuit and a receiving wave circuit (not illustrated). The transmitting wave circuit transmits the pulse waves to the ultrasound transceiver 310 inside the imaging core 220 based on a control signal transmitted from the signal processing unit 428.

In addition, the receiving wave circuit receives an ultrasound signal from the ultrasound transceiver 310 inside the imaging core 220. The received ultrasound signal can be amplified by an amplifier 453, and then is input to and detected by a wave detector 454.

Furthermore, an A/D converter 455 generates digital data (ultrasound data) of one line by sampling the ultrasound signal output from the wave detector 454, for example, at 30.6 MHz by an amount of 200 points. Although 30.6 MHz is used here, this is calculated on the assumption that the sampling of 200 points is performed for a depth of 5 mm when sound velocity is set to 1530 m/sec. Therefore, the sampling frequency is not particularly limited thereto.

The ultrasound data in units of lines, which is generated by the A/D converter 455 is input to the signal processing unit 428. The signal processing unit 428 converts the ultrasound data into a gray scale, thereby generating an ultrasound tomographic image at each position inside a blood vessel and outputting the ultrasound cross-sectional image to an LCD monitor 113 at a predetermined frame rate.

The signal processing unit 428 is connected to a motor control circuit 429, and receives a video synchronization signal of the motor control circuit 429. The signal processing unit 428 generates the ultrasound tomographic image in synchronization with the received video synchronization signal.

In addition, the video synchronization signal of the motor control circuit 429 is also transmitted to the rotary drive device 404, and the rotary drive device 404 outputs a drive signal synchronized with the video synchronization signal.

In accordance with an exemplary embodiment, in order to output a generated ultrasound tomographic image to the LCD monitor 113, a correction value for correcting a distance difference in an axial direction and/or an angular difference in a circumferential direction between the ultrasound transceiver 310 and the optical transceiver 320 which is calculated by performing a calibration process using a calibration tool (to be described later) is used, thereby outputting the ultrasound tomographic image subjected to position correction.
(2) Function of Wavelength Swept-Type OCT

Next, a functional configuration of wavelength swept-type OCT will be described with reference to the same drawings. The reference numeral 408 represents a wavelength swept light source (swept laser), and is one type of an extended-cavity laser which can include an optical fiber 416 which is coupled to a semiconductor optical amplifier (SOA) 415 in a ring shape and a polygon scanning filter (408b).

Light output from the SOA 415 moves forward to the optical fiber 416, and enters the polygon scanning filter 408b. The light whose wavelength is selected here is amplified by the SOA 415, and is finally output from a coupler 414.

The polygon scanning filter 408b selects the wavelength in combination with a diffraction grating 412 for diffracting the light and a polygon mirror 409. In accordance with an exemplary embodiment, the light diffracted by the diffraction grating 412 can be concentrated on a surface of the polygon mirror 409 by two lenses (410 and 411). In this manner, only the light having a wavelength orthogonal to the polygon mirror 409 returns through the same optical path, and is output from the polygon scanning filter 408b. That is, time sweeping of the wavelength can be performed by rotating the polygon mirror 409.

For example, a 32-sided mirror can be used for the polygon mirror 409 whose rotation speed, can be, for example, approximately 50000 rpm. A wavelength swept system in which the polygon mirror 409 and the diffraction grating 412 can be combined with each other, which can help enable high speed and high output wavelength sweeping.

The light of a wavelength swept light source 408 which is output from the coupler 414 is incident on one end (proximal end) of a first single mode fiber 440, and is transmitted to the distal end side of the first single mode fiber 440. The first single mode fiber 440 can be optically coupled to a second single mode fiber 445 and a third single mode fiber 444 in an optical coupler 441 located in the middle therebetween.

In accordance with an exemplary embodiment, on the further distal end side than the optical coupler 441 of the first single mode fiber 440, an optical rotary joint (optical coupling unit) 403 which can transmit the light by coupling a non-rotating part (fixed portion) and a rotating part (rotary drive unit) to each other is disposed inside the rotary drive device 404.

Furthermore, a fifth single mode fiber 443 of the probe unit 101 can be detachably connected via the adapter 402 to the distal end side of a fourth single mode fiber 442 inside the optical rotary joint (optical coupling unit) 403. In this manner, the light from the wavelength swept light source 408 can be transmitted to the fifth single mode fiber 443 which is inserted into the imaging core 220 and can be rotatably driven.

The transmitted light is emitted from the optical transceiver 320 of the imaging core 220 to the biological tissues inside the blood vessel while a rotary operation and an axial operation are performed. Then, the reflected light scattered on a surface or inside the biological tissues is partially captured by the optical transceiver 320 of the imaging core 220, and returns to the first single mode fiber 440 side through a rearward optical path. Furthermore, the light is partially transferred to the second single mode fiber 445 side by the optical coupler 441, and is emitted from one end of the second single mode fiber 445. Thereafter, the light is received by an optical detector (for example, a photodiode 424).

The rotary drive unit side of the optical rotary joint 403 is rotatably driven by the radial scanning motor 405 of the rotary drive device 404.

In accordance with an exemplary embodiment, an optical path length variable mechanism 432 for finely adjusting an optical path length of reference light can be disposed in the distal end opposite to the optical coupler 441 of the third single mode fiber 444.

In order for variations in the length of an individual probe unit 101 to be absorbed when the probe unit 101 is replaced and newly used, the optical path length variable mechanism 432 can include optical path length changing means for changing an optical path length corresponding to the variations in the length.

The third single mode fiber 444 and a collimating lens 418 can be disposed on a one-axis stage 422 which is movable in an optical axis direction thereof as illustrated by an arrow 423, thereby forming the optical path length changing means.

In accordance with an exemplary embodiment, the one-axis stage 422 functions as the optical path length changing means having a variable enough range of the optical path length to absorb the variations in the optical path length of the probe unit 101 when the probe unit 101 is replaced. Furthermore, the one-axis stage 422 can also include an adjusting means for adjusting an offset. For example, even when the distal end of the probe unit 101 is not in close contact with the surface of the biological tissues, the one-axis stage can finely change the optical path length. In this manner, the optical path length can be set in a state of interfering with the reflected light from the surface position of the biological tissues.

The optical path length is finely adjusted by the one-axis stage 422. The light reflected on a mirror 421 via a grating 419 and a lens 420 is mixed with the light obtained from the first single mode fiber 440 side by the optical coupler 441 disposed in the middle of the third single mode fiber 444, and then is received by the photodiode 424.

Interference light received by the photodiode 424 in this way can be photoelectrically converted, and can be input to a demodulator 426 after being amplified by the amplifier 425. The demodulator 426 performs demodulation processing for extracting only a signal portion of the interference light, and an output therefrom is input to the A/D converter 427 as an interference light signal.

The A/D converter 427 performs sampling on the interference light signal, for example, at 180 MHz by an amount of 2048 points, and generates digital data (interference light data) of one line. In accordance with an exemplary embodiment, the reason for setting the sampling frequency to 180 MHz is on the assumption that approximately 90% of wavelength swept cycles (12.5 μsec) is extracted as the digital data of 2048 points, when a repetition frequency of the wavelength sweeping is set to 80 kHz. However, the sampling frequency is not particularly limited thereto.

The interference light data in the units of lines, which is generated by the A/D converter 427 is input to the signal processing unit 428. The signal processing unit 428 generates data in a depth direction (line data) by performing frequency resolution on the interference light data using the fast Fourier transform (FFT), and the data is subjected to coordinate transformation. In this manner, an optical cross-sectional image is constructed at each intravascular position, and is output to the LCD monitor 113 at a predetermined frame rate.

The signal processing unit 428 can be further connected to a control device of optical path length adjusting means 430. In addition, the signal processing unit 428 can control a position of the one-axis stage 422 via the control device of optical path length adjusting means 430.

### 5. Description of Signal Processing Unit

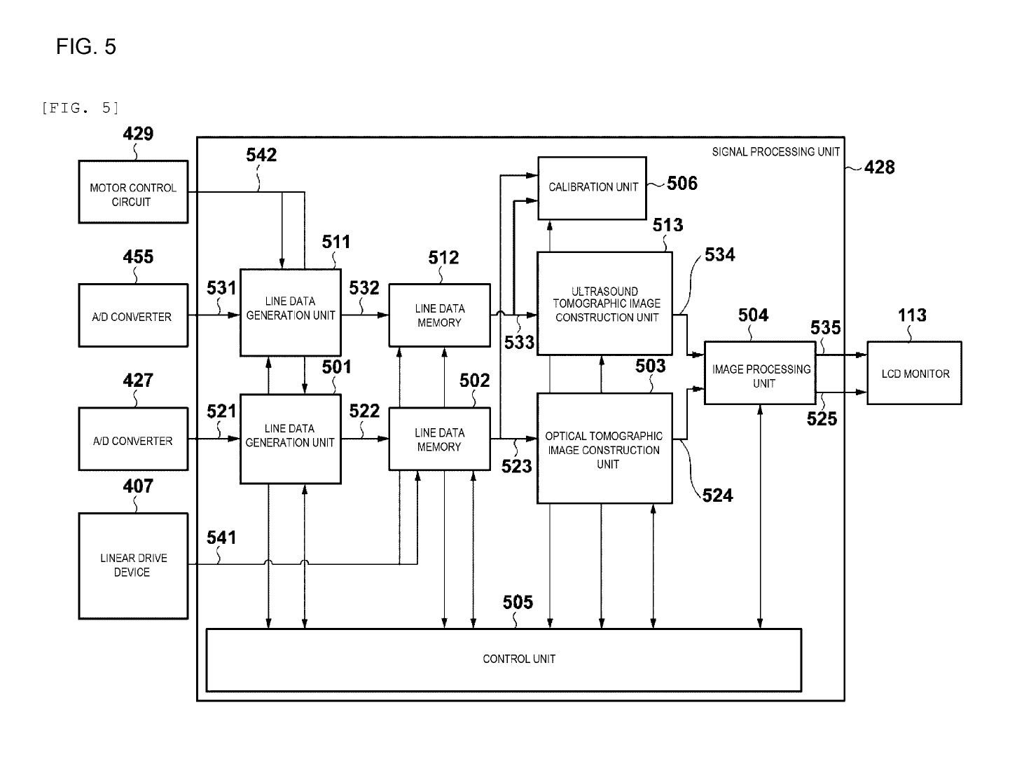

Next, a functional configuration of the signal processing unit 428 of the imaging apparatus for diagnosis 100 will be described. FIG. 5 is a diagram illustrating the functional configuration and the related functional blocks of the signal processing unit 428 of the imaging apparatus for diagnosis 100. The functional configuration illustrated in FIG. 5 may be realized by using dedicated hardware, or may be partially realized by software (that is, by causing a computer to execute a program for realizing the function).

As illustrated in FIG. 5, interference light data 521 generated in the A/D converter 427 is processed in the line data generation unit 501 inside the signal processing unit 428 by using a signal of the encoder unit 406 of the radial scanning motor 405 which is output from the motor control circuit 429 so that the number of lines per one rotation is 512.

The line data 522 output from the line data generation unit 501 is stored in a line data memory 502 per one rotation (one frame), based on an instruction from the control unit 505. At this time, the control unit 505 counts pulse signals 541 output from a movement amount detector of the linear drive apparatus 407. When the line data 522 is stored in the line data memory 502, the control unit 505 stores respective items of the line data 522 in association with a count value when the line data 522 is generated.

Line data 523 stored in association with the count value is input to a calibration unit 506 in a calibration mode for performing a calibration process using the calibration tool (to be described later), based on the instruction from the control unit 505. In addition, in a generation mode for generating the optical tomographic image, based on the instruction from the control unit 505, the line data 523 is subjected to Rθ conversion after various processes (line averaging process, filtering process or the like) are performed in an optical tomographic image construction unit 503, and is sequentially output as an optical tomographic image 524.

Furthermore, after image processing is performed to be displayed on the LCD monitor 113 in an image processing unit 504, the line data 523 is output to the LCD monitor 113 as an optical tomographic image 525.

Similarly, ultrasound data 531 generated in the A/D converter 455 is processed in a line data generation unit 511 inside the signal processing unit 428 by using a signal of the encoder unit 406 of the radial scanning motor 405 which is output from the motor control circuit 429 so that the number of lines per one rotation is saved in a line data memory 512.

The line data 532 output from the line data generation unit 511 is stored in the line data memory 512 per one rotation (one frame), based on an instruction from the control unit 505. At this time, the control unit 505 counts the pulse signals 541 output from the movement amount detector of the linear drive apparatus 407. When the line data 532 is stored in the line data memory 512, the control unit 505 stores respective items of the line data 532 in association with the count value when the line data 532 is generated.

Line data 533 stored in association with the count value is input to the calibration unit 506 in the calibration mode, based on the instruction from the control unit 505. In addition, in a generation mode for generating the ultrasound tomographic image, based on the instruction from the control unit 505, the line data 533 is subjected to Re conversion after various processes (line averaging process, filtering process or the like) are performed in an ultrasound tomographic image construction unit 513, and is sequentially output as an ultrasound tomographic image 534.

In accordance with an exemplary embodiment, after image processing is performed to be displayed on the LCD monitor 113 in the image processing unit 504 and position correction processing is performed by using a correction value (correction value for aligning the ultrasound tomographic image and the optical tomographic image with each other) calculated in the calibration unit 506, the line data 533 is output to the LCD monitor 113 as the ultrasound tomographic image 534.

### 6. Description of Calibration Tool

Next, the calibration tool 600 for calculating the distance difference in the axial direction and the angular difference in the circumferential direction between the transmitting and receiving direction of the ultrasound transceiver 310 and the transmitting and receiving direction of the optical transceiver 320 will be described. In order to simplify description in the present embodiment, the distance difference in the axial direction between the transmitting and receiving direction of the ultrasound transceiver 310 and the transmitting and receiving direction of the optical transceiver 320 is assumed to be known. Accordingly, the calibration tool used to calculate only the angular difference in the circumferential direction will be described.

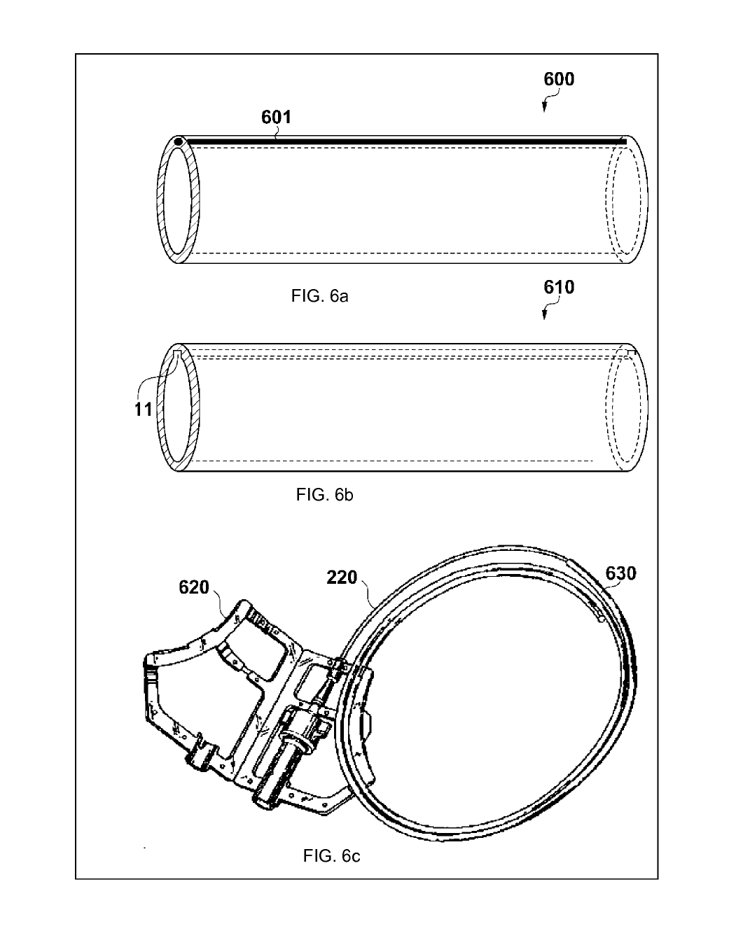

FIGS. 6A-6C are view illustrating the calibration tool 600 used to calculate the angular difference in the circumferential direction between the transmitting and receiving direction of the ultrasound transceiver 310 and the transmitting and receiving direction of the optical transceiver 320. As illustrated in FIGS. 6A and 6B, the calibration tool 600 has a hollow cylindrical shape, and has a configuration in which the imaging core 220 can be inserted into the calibration tool. The calibration tool 600 may be configured to serve as a dedicated tool. As illustrated in FIG. 6C, when the imaging core 220 is delivered by being fixed to a holder 620, the calibration tool 600 may be realized in a hollow cylindrical protection member 630, which is mounted to protect the imaging core 220.

Among the calibration tools, in a case of a calibration tool 600 illustrated in FIG. 6A, a linear reflection section 601 arranged substantially parallel to the axial direction is arranged on an inner wall surface or an outer wall surface therein. For example, the reflection section 601 is formed of aluminum, which can cause ultrasound waves transmitted from the ultrasound transceiver 310 and light transmitted from the optical transceiver 320 to be reflected on the reflection section 601. In accordance with an exemplary embodiment, the material of the reflection section 601 is not limited to the aluminum, and may be a material, which is different from a material of a wall surface of the calibration tool 600.

In accordance with an exemplary embodiment, in a case of the calibration tool 600 illustrated in FIG. 6B, a linear groove 611 arranged substantially parallel to the axial direction is arranged on the inner wall surface therein. In this way, disposing the groove in a portion on the inner wall surface allows the ultrasound waves transmitted from the ultrasound transceiver 310 and the light transmitted from the optical transceiver 320 to be reflected on the groove 611. In accordance with an exemplary embodiment, for example, the groove 611 is included in the reflection section in a broad sense.

7. Operation of Imaging Core when Calibration Is Performed Using Calibration Tool

Next, a relationship between an operation of the imaging core 220 and line data acquired by the operation of the imaging core 220 when calibration is performed using the calibration tool 600 (or 601) will be described.

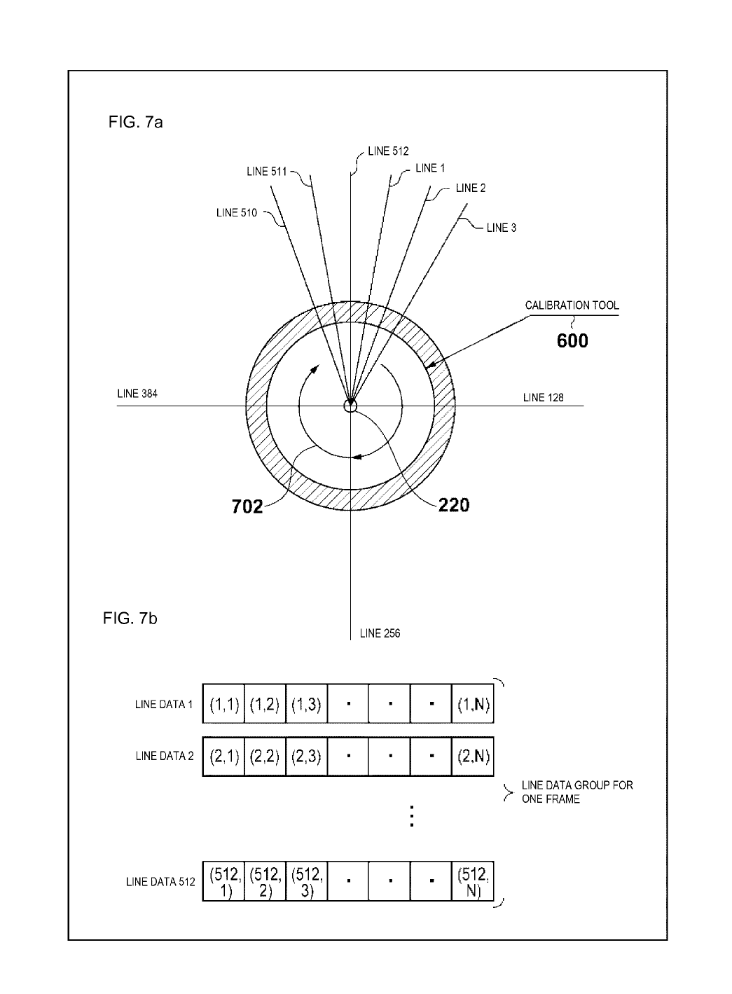

FIG. 7A illustrates a state where the imaging core 220 inserted into the calibration tool 600 during the calibration process is viewed from an opening side of the calibration tool 600. If the calibration process starts in this state, the imaging core 220 is rotated in a direction of an arrow 702 by the radial scanning motor 405.

At this time, the ultrasound transceiver 310 transmits and receives the ultrasound waves at each rotation angle. Lines 1, 2 to 512 illustrate the transmitting and receiving directions of the ultrasound waves at each rotation angle. In the imaging apparatus for diagnosis 100 according to the present embodiment, the ultrasound transceiver 310 intermittently transmits and receives the ultrasound waves 512 times while the ultrasound transceiver 310 is rotated by 360 degrees inside the calibration tool 600.

Similarly, the optical transceiver 320 also transmits and receives the light at each rotation angle. The optical transceiver 320 continuously transmits and receives the light 512 times while the optical transceiver 320 is rotated by 360 degrees inside the calibration tool 600.

In FIG. 7A, the transmitting and receiving direction of the light is not illustrated. However, since the optical transceiver 320 and the ultrasound transceiver 310 are arranged to have an angular difference in the circumferential direction, the transmitting and receiving direction of the light is not coincident with the transmitting and receiving direction of the ultrasound waves. For example, the direction of the line 1 in the ultrasound transceiver 310 is not the same as the direction of the line 1 (not illustrated) in the optical transceiver 320.

FIG. 7B illustrates a configuration of the line data obtained by transmitting and receiving the ultrasound waves or the light at each rotation angle. As illustrated in FIG. 7B, one frame of the ultrasound tomographic image and one frame of the optical tomographic image in the present embodiment are respectively configured to include a line data group having 512 lines. Each line data has a data group having the N number of pixels in the transmitting and receiving direction of the ultrasound waves or the light (for example, the N number is 1024).

The ultrasound waves and the light are transmitted and received while advancing in the axial direction inside the calibration tool 600. Accordingly, the data for the ultrasound tomographic image and the data for the optical tomographic image, which include the line data group illustrated in FIG. 7B, are generated by multiple frames in the axial direction.

### 8. Example of Data for Ultrasound Tomographic Image and Data for Optical Tomographic Image

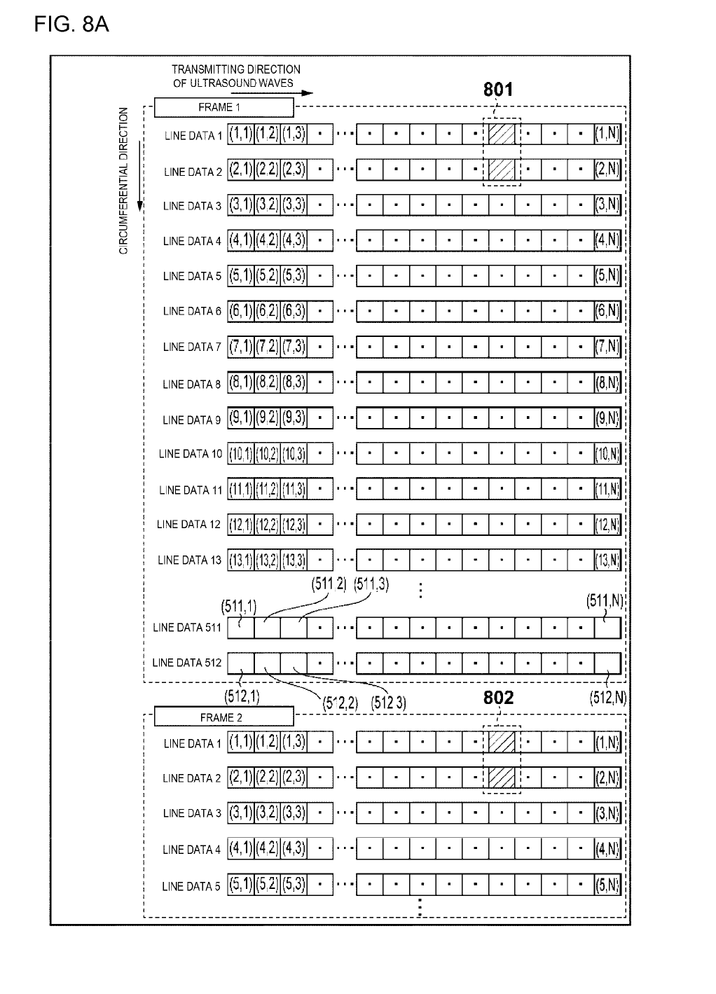

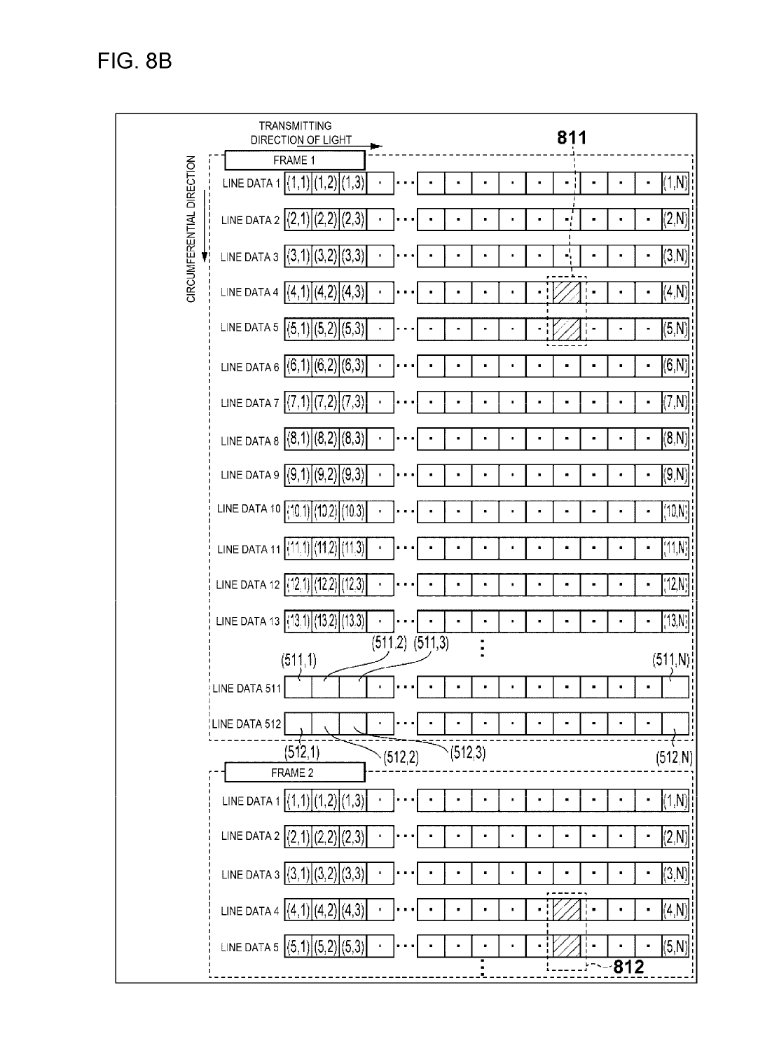

Next, an example of data for ultrasound tomographic image and data for optical tomographic image, which are acquired when the calibration process is performed by using the calibration tool 600 will be described. FIGS. 8A and 8B are diagrams illustrating an example of the data for ultrasound tomographic image and the data for optical tomographic image which are obtained by the ultrasound transceiver 310 transmitting and receiving the ultrasound waves and the optical transceiver 320 transmitting and receiving the light in a state where the imaging core 220 inserted into the calibration tool 600 is moved in the axial direction while being rotated in the circumferential direction.

In FIG. 8A, a portion where pixel data 801 is hatched illustrates the reflection section 601 of the calibration tool 600, which is detected by the rotary operation in which the ultrasound transceiver 310 is rotated for the first time in the circumferential direction. In addition, a portion where pixel data 802 is hatched illustrates the reflection section 601 of the calibration tool 600, which is detected by the rotary operation in which the ultrasound transceiver 310 is rotated for the second time in the circumferential direction.

The reflection section 601 arranged in the calibration tool 600 is formed in parallel with the axial direction and in a linear shape. Accordingly, the reflection section 601 is detected at the same position of each frame.

Similarly, in FIG. 8B, a portion where pixel data 811 is hatched illustrates the reflection section 601 of the calibration tool 600 which is detected by the rotary operation in which the optical transceiver 320 is rotated for the first time in the circumferential direction. In addition, a portion where pixel data 812 is hatched illustrates the reflection section 601 of the calibration tool 600, which is detected by the rotary operation in which the optical transceiver 320 is rotated for the second time in the circumferential direction.

As described above, the reflection section 601 arranged in the calibration tool 600 is formed in parallel with the axial direction and in the linear shape. Accordingly, the reflection section 601 is detected at the same position of each frame. However, since the ultrasound transceiver 310 and the optical transceiver 320 are arranged to have an angular difference in the circumferential direction, a detection position of the reflection section 601 in each frame of the data for ultrasound tomographic image and a detection position of the reflection section 601 in each frame of the data for optical tomographic image are not the same as each other, and are deviated from each other in the circumferential direction.

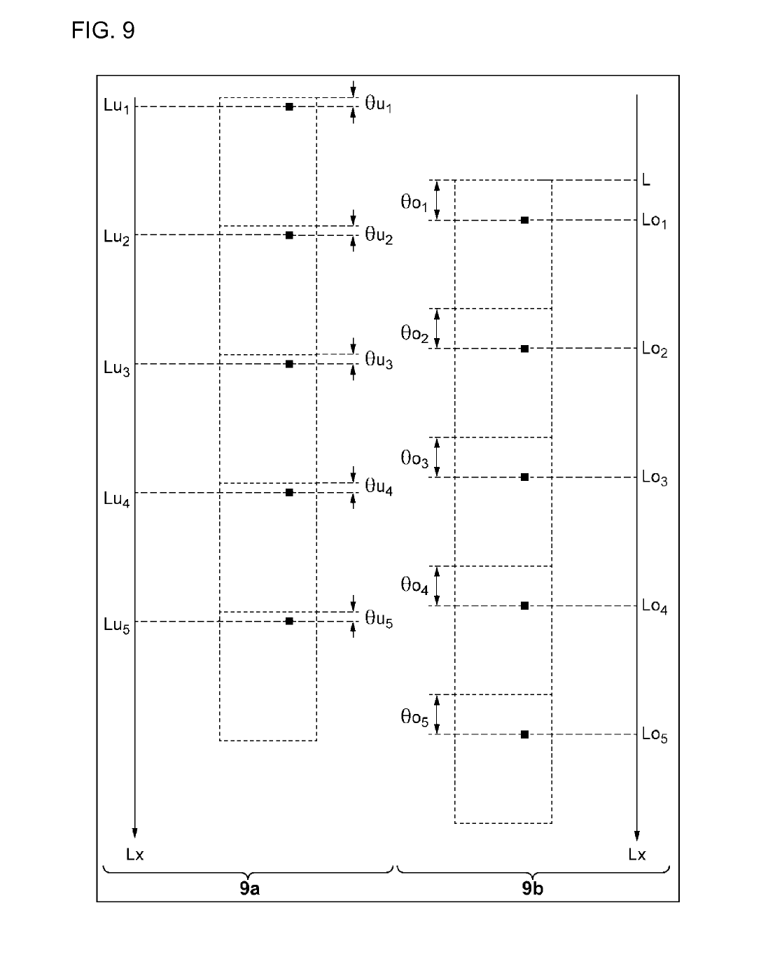

FIG. 9 is a diagram schematically illustrating a case where the position of detecting the reflection section 601 in each frame of the data for ultrasound tomographic image is juxtaposed with the position of detecting the reflection section 601 in each frame of the data for optical tomographic image.

In portion 9a of FIG. 9A, the reference numeral θᵤ₁ represents an angle between line data from which the reflection section 601 is detected and line data 1 (frame end) in the first frame. The reference numeral θᵤ₂ represents an angle between the line data from which the reflection section 601 is detected and the line data 1 (frame end) in the second frame. In the following, similarly, the reference numerals θᵤ₃, θᵤ₄, and θᵤ₅ respectively represent angles between the line data from which the reflection section 601 is detected and the line data 1 (frame end) in the third, fourth, and fifth frames.

In addition, the reference numeral Lᵤ₁ represents a position in the axial direction at which the reflection section 601 is detected in the first frame, based on the position before the ultrasound transceiver 310 starts to move in the axial direction (equal to the distance corresponding to a count value obtained by counting the pulse signals 541 output from the movement amount detector of the linear drive apparatus 407). In addition, the reference numeral Lᵤ₂ represents the position in the axial direction at which the reflection section 601 is detected in the second frame. In the following, similarly, the reference numerals Lᵤ₃, Lᵤ₄, and Lᵤ₅ respectively represent positions in the axial direction at which the reflection section 601 is detected in the third, fourth, and fifth frames.

Similarly, in portion 9b of FIG. 9, the reference numeral θₒ₁ represents the angle between line data from which the reflection section 601 is detected and the line data 1 (frame end) in the first frame. The reference numeral θₒ₂ represents the angle between the line data from which the reflection section 601 is detected and the line data 1 (frame end) in the second frame. In the following, similarly, the reference numerals θₒ₃, θₒ₄, and θₒ₅ respectively represent the angles between the line data from which the reflection section 601 is detected and the line data 1 (frame end) in the third, fourth, and fifth frames.

In addition, the reference numeral Lₒ₁ represents the position in the axial direction at which the reflection section 601 is detected in the first frame, based on the position before the ultrasound transceiver 310 starts to move in the axial direction (equal to a value obtained by adding a distance L in the axial direction between the ultrasound transceiver 310 and the optical transceiver 320 to the distance corresponding to the count value obtained by counting the pulse signals 541 output from the movement amount detector of the linear drive apparatus 407). As described above, the optical transceiver 320 is arranged at a position separated to the further proximal end side by the distance L as compared to the ultrasound transceiver 310. Accordingly, the position in the axial direction of the first frame of the data for ultrasound tomographic image and the position in the axial direction of the first frame of the data for optical tomographic image are deviated from each other by the distance L. In addition, the reference numeral Lᵤ₂ represents the position in the axial direction at which the reflection section 601 is detected, in the second frame, and the reference numerals Lᵤ₃, L_{U4}, and Lᵤ₅ respectively represent the positions in the axial direction at which the reflection section 601 is detected in the third, fourth, and fifth frames.

### 9. Calibration Process in Calibration Unit

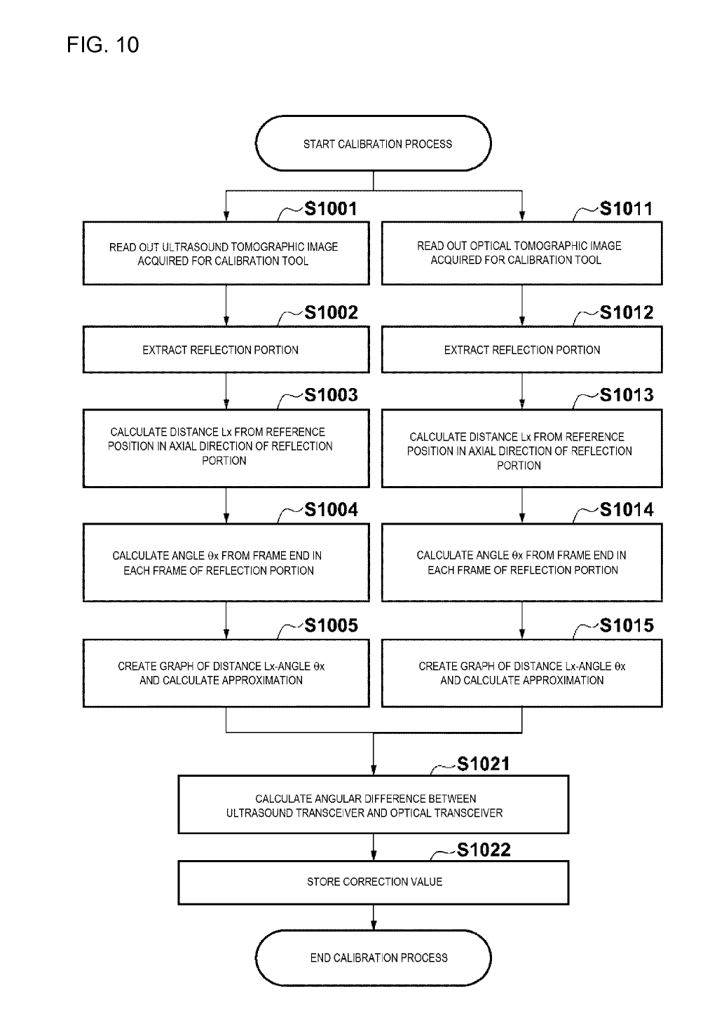

Next, a calibration process in the calibration unit 506 will be described. FIG. 10 is a flowchart illustrating flow in the calibration process in the calibration unit 506.

In accordance with an exemplary embodiment, if a user starts the calibration process by selecting a calibration mode in a state where the imaging core 220 is inserted into the calibration tool 600, the data for ultrasound tomographic image and the data for optical tomographic image are acquired with respect to the calibration tool 600. If the data for ultrasound tomographic image and the data for optical tomographic image are completely acquired so as to reach a predetermined data amount, the calibration process illustrated in FIG. 10 starts.

In Step S1001, the data for ultrasound tomographic image acquired with respect to the calibration tool 600 is read out. In Step S1002, the reflection section 601 is extracted from each frame.

Furthermore, in Step S1003, distances Lx from a reference position in the axial direction to the reflection sections 601 extracted from each frame in Step S1002 are respectively calculated. In addition, in Step S1004, angles θx between the frame end (line data 1) in each frame and the reflection sections 601 extracted from each frame in Step S1002 are respectively calculated.

In Step S1005, a graph is created in which the distance Lx is set to the horizontal axis and the angle θx is set to the vertical axis. Then, a value calculated in Step S1003 and Step S1004 is plotted on the graph. In addition, an approximation is calculated with regard to the plotted result.

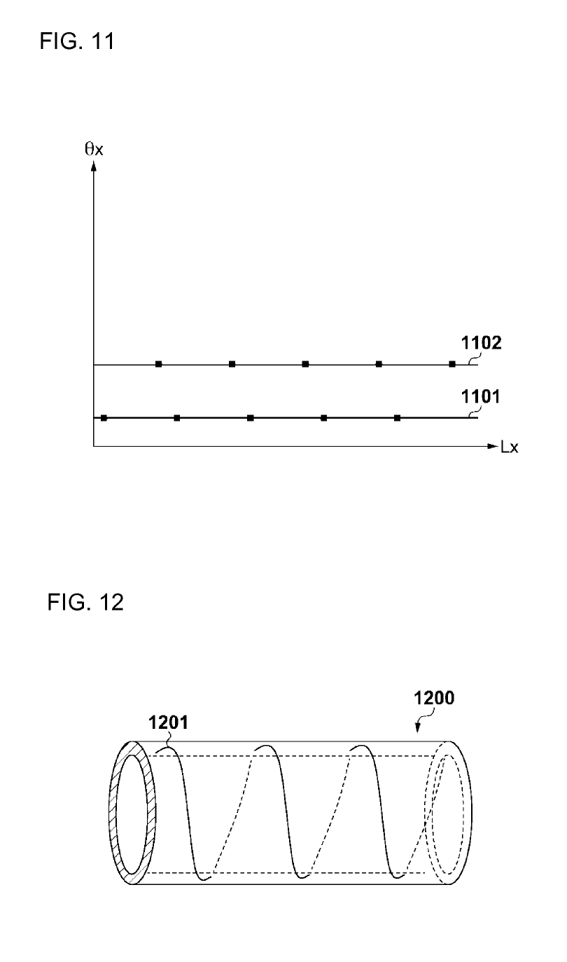

FIG. 11 is a graph in which the distance Lx is set to the horizontal axis and the angle θx is set to the vertical axis, and the reference numeral 1101 illustrates the approximation calculated by plotting (Lᵤ₁, θᵤ₁), (Lᵤ₂, θᵤ₂), (Lᵤ₃, θᵤ₃), (Lᵤ₄, θᵤ₄), and (Lᵤ₅, θᵤ₅) which are calculated in Step S1003 and Step S1004.

Referring back to FIG. 10, the process will be described. In Step S1011, the data for optical tomographic image acquired with respect to the calibration tool 600 is read out. In Step S1012, the reflection section 601 is extracted from each frame.

Furthermore, in Step S1013, the distances Lx from the reference position in the axial direction to the reflection sections 601 extracted from each frame in Step S1012 are respectively calculated. In addition, in Step S1014, the angles Ox between the frame end (line data 1) in each frame and the reflection sections 601 extracted from each frame in Step S1012 are respectively calculated.

In Step S1015, a graph is created in which the distance Lx is set to the horizontal axis and the angle θx is set to the vertical axis. Then, a value calculated in Step S1013 and Step S1014 is plotted in the graph. In addition, based on the plotted result, approximation is calculated.

In FIG. 11, the reference numeral 1102 illustrates approximation calculated by plotting (Lₒ₁, θₒ₁), (Lₒ₂, θₒ₂), (Lₒ₃, θₒ₃), (Lₒ₄, θₒ₄), and (Lₒ₅, θₒ₅) which are calculated in Step S1013 and Step S1014.

Referring back to FIG. 10, the process will be described. In Step S1021, based on an approximation 1101 calculated in Step S1005 and an approximation 1102 calculated in Step S1015, an angular difference in the circumferential direction is calculated between the transmitting and receiving direction of the ultrasound transceiver 310 and the transmitting and receiving direction of the optical transceiver 320. In accordance with an exemplary embodiment, the angular difference can be calculated in the circumferential direction by comparing the intercept intersecting the Ox axis of the approximation 1101 with the intercept intersecting the Ox axis of the approximation 1102.

In Step S1022, the angular difference in the circumferential direction, which is calculated in Step S1021, is stored in the signal processing unit 428 as a correction value for a position correction process when the ultrasound tomographic image 535 is output to the LCD monitor 113, and then the calibration process ends.

As is apparent from the above description, the present embodiment adopts a configuration in which the angular difference in the circumferential direction between the ultrasound transceiver and the optical transceiver is calculated by using the calibration tool, which is formed in the hollow cylindrical shape and has the linear reflection section substantially parallel to the axial direction.

In accordance with an exemplary embodiment, a configuration is adopted in which positional information of the reflection section (distance from the reference position in the axial direction, angle from each frame end) is obtained from the data for ultrasound tomographic image and the data for optical tomographic image with respect to the calibration tool by providing a calibration mode in the imaging apparatus for diagnosis.

In addition, a configuration is adopted in which the angular difference in the circumferential direction between the ultrasound transceiver and the optical transceiver is calculated by plotting the position of the reflection section of each frame on the graph in which the distance from the reference position in the axial direction and the angle from each frame end are respectively set to the horizontal axis and the vertical axis so as to calculate the equation.

Furthermore, a configuration is adopted in which the calculated angular difference is used as the correction value for position correction when the ultrasound tomographic image is output to the LCD monitor.

As a result, even when the angular difference in the circumferential direction between the ultrasound transceiver and the optical transceiver is not known, the position correction process can be performed according to the angular difference by performing the calibration process using the calibration tool.

In the above-described first embodiment, a case has been described where the reflection section 601 is arranged substantially parallel to the axial direction as the calibration tool. However, the present disclosure is not limited thereto. For example, the reflection section may be arranged in a spiral shape. Hereinafter, details of the present embodiment will be described.
(1) Description of Calibration Tool

First, the calibration tool used for the calibration process of the imaging apparatus for diagnosis 100 according to the present embodiment will be described. FIG. 12 is a view illustrating an example of a calibration tool 1200 according to the present embodiment. In order to simplify the description in the present embodiment, the distance difference in the axial direction between the transmitting and receiving direction of the ultrasound transceiver 310 and the transmitting and receiving direction of the optical transceiver 320 is also assumed to be known. Accordingly, the calibration tool is assumed to calculate only the angular difference in the circumferential direction.

As illustrated in FIG. 12, a reflection section 1201 is arranged in a spiral shape on an outer peripheral surface of the calibration tool 1200 with a constant pitch in the axial direction. Similarly to the above-described first embodiment, the reflection section 1201 is formed of aluminum, for example, which can cause the ultrasound waves transmitted from the ultrasound transceiver 310 and the light transmitted from the optical transceiver 320 to be reflected on the reflection section 1201. A material of the reflection section 1201 is not limited to the aluminum, and may be a material, which is different from a material of a wall surface of the calibration tool 1200.

In accordance with an exemplary embodiment, the spirally wound direction of the reflection section 1201 can be different from the rotation direction of the imaging core 220 since this can reliably detect the reflection section 1201.
(2) Example of Data for Ultrasound Tomographic Image and Data for Optical Tomographic Image

Next, an example of the data for ultrasound tomographic image and the data for optical tomographic image which are acquired when the calibration process is performed by using the calibration tool 1200 will be described.

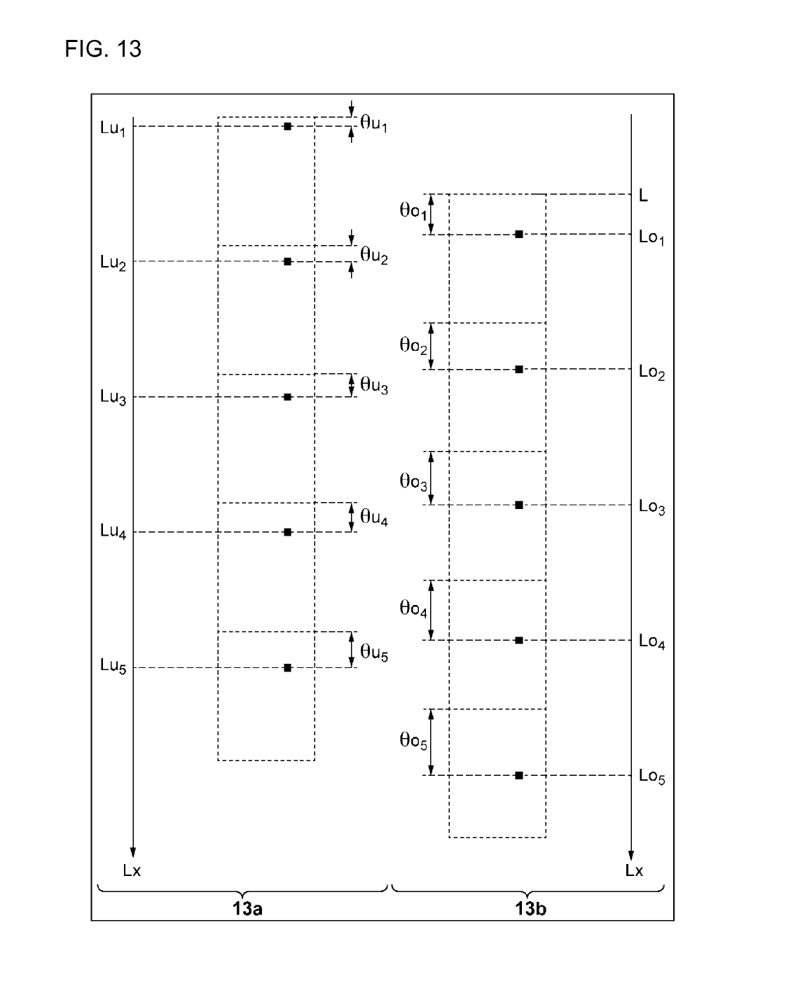

FIG. 13 is a diagram schematically illustrating the position at which the reflection section 1201 is detected in each frame of the data for ultrasound tomographic image and the position at which the reflection section 1201 is detected in each frame of the data for optical tomographic image.

The reference numerals θᵤ₁ to θᵤ₅ and Lᵤ₁ to Lᵤ₅ in portion 13a of FIG. 13 have been described with reference to portion 9a of FIG. 9 in the above-described first embodiment. Thus, description thereof will be omitted here.

In addition, the reference numerals θₒ₁ to θₒ₅ and Lₒ₁ to Lₒ₅ in portion 13b of FIG. 13 have been described with reference to portion 9b of FIG. 9 in the above-described exemplary embodiment. Thus, description thereof will be omitted here.

As illustrated in portions 13a and 13b of FIG. 13, in a case of the calibration tool 1200, the reflection section 1201 is arranged in a spiral shape. Accordingly, an angle from the frame end to the reflection section 1201 is not constant in each frame, and gradually increases as the frame moves forward.

### 10. Calibration Process in Calibration Unit

Next, the calibration process in the calibration unit 506 will be described. Flow in the calibration process in the calibration unit 506 is the same as that in FIG. 10. However, in the graph created in Step S1005, the plotted result and the calculated approximation when the calculated value in Step S1003 and Step S1004 is plotted are different therefrom. Similarly, in the graph created in Step S1015, the plotted result and the calculated approximation when the calculated value in Step S1013 and Step S1014 is plotted are different therefrom.

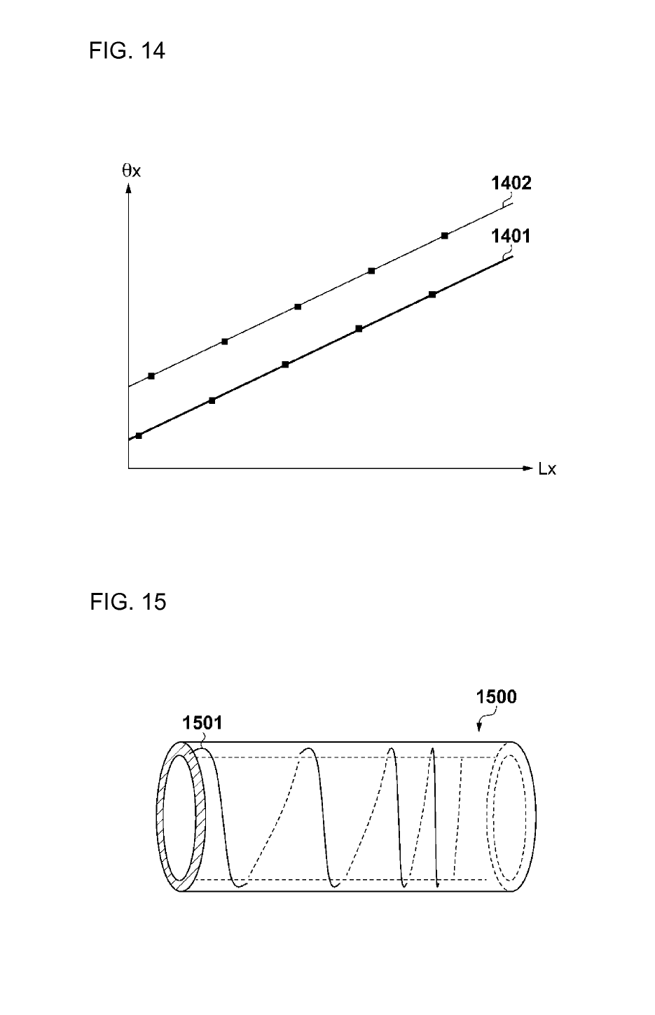

FIG. 14 is a view illustrating the graph and the approximation, which are created by performing the calibration process using the calibration tool 1200.

In FIG. 14, the reference numeral 1401 illustrates the approximation calculated by plotting (Lᵤ₁, θᵤ₁), (Lᵤ₂, θᵤ₂), (Lᵤ₃, θᵤ₃), (Lᵤ₄, θᵤ₄), and (Lᵤ₅, θᵤ₅) which are calculated in Step S1003 and Step S1004 with respect to the calibration tool 1200. In addition, the reference numeral 1402 illustrates the approximation calculated by plotting (Lₒ₁, θₒ₁), (Lₒ₂, θₒ₂), (Lₒ₃, θₒ₃), (Lₒ₄, θₒ₄), and (Lₒ₅, θₒ₅) which are calculated in Step S1013 and Step S1014 with respect to the calibration tool 1200.

As illustrated in FIG. 14, when the calibration process is performed by using the calibration tool 1200 in which the reflection section 1201 is arranged in a spiral shape, the approximations 1401 and 1402 come to have a predetermined inclination with respect to the horizontal axis. A method of calculating the angular difference in the circumferential direction using the approximations 1401 and 1402 is the same as that in the above-described first embodiment. In accordance with an exemplary embodiment, the angular difference can be calculated in the circumferential direction by comparing the intercept intersecting the θx axis of the approximation 1401 with the intercept intersecting the θx axis of the approximation 1402.

As is apparent from the above description, the present embodiment adopts a configuration in which the angular difference in the circumferential direction between the ultrasound transceiver and the optical transceiver is calculated by using the calibration tool, which is formed in the hollow cylindrical shape, and has the reflection section arranged in a spiral shape.

As a result, even when the angular difference in the circumferential direction between the ultrasound transceiver and the optical transceiver is not known, the position correction process can be performed according to the angular difference by performing the calibration process using the calibration tool.

In the above-described first and second embodiments, a case has been described where the position correction process can be performed according to the angular difference in the circumferential direction by performing the calibration process using the calibration tool 600 or 1200, when the distance difference in the axial direction between the transmitting and receiving direction of the ultrasound transceiver 310 and the transmitting and receiving direction of the optical transceiver 320 is known and the angular difference in the circumferential direction is not known.

However, the present disclosure is not limited thereto. For example, even when both the distance difference in the axial direction and the angular difference in the circumferential direction between the transmitting and receiving direction of the ultrasound transceiver 310 and the transmitting and receiving direction of the optical transceiver 320 are not known, both of these can be calculated by performing the similar calibration process depending on a shape of the calibration tool. Hereinafter, details of the present embodiment will be described.
(1) Description of Calibration Tool

FIG. 15 is a view illustrating a calibration tool 1500 used when the imaging apparatus for diagnosis 100 according to the present embodiment performs the calibration process. As illustrated in FIG. 15, the calibration tool 1500 has a hollow cylindrical shape, and a reflection section 1501 is arranged in a spiral shape on an outer peripheral surface of the calibration tool 1500.

In the above-described second embodiment, in a case of the calibration tool 1200 illustrated in FIG. 12, the reflection section 1201 is arranged on the outer peripheral surface at a constant pitch in the axial direction. In contrast, in a case of the calibration tool 1500 according to the present embodiment, the pitch in the axial direction of the reflection section 1501 is not constant. In accordance with an exemplary embodiment, the calibration tool 1200 described with reference to FIG. 12 is different since the reflection section 1501 is arranged so that the pitch is gradually widened as it moves forward in the axial direction.
(2) Example of Data for Ultrasound Tomographic Image and Data for Optical Tomographic Image

Next, an example of the data for ultrasound tomographic image and the data for optical tomographic image which are acquired when the calibration process is performed by using the calibration tool 1500 will be described.

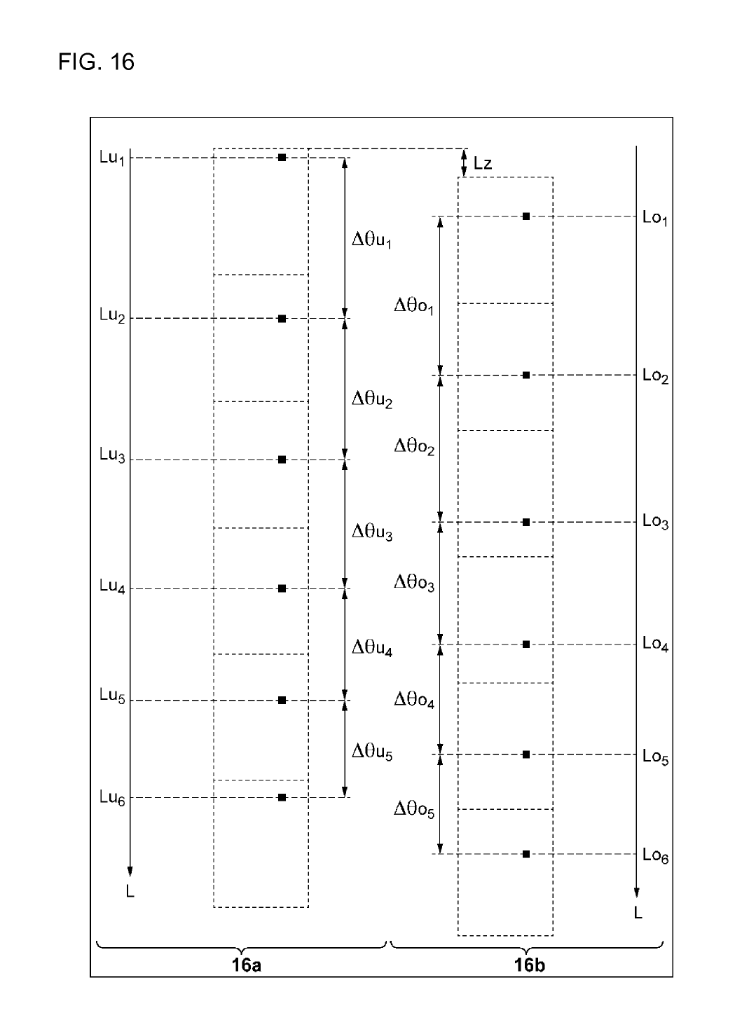

FIG. 16 is a diagram schematically illustrating a position at which the reflection section 1501 is detected in each frame of the data for ultrasound tomographic image and a position at which the reflection section 1501 is detected in each frame of the data for optical tomographic image.

In portion 16a of FIG. 16, the reference numeral Δθᵤ₁ represents an angular difference between a position in the circumferential direction at which the reflection section 1501 is detected in the first frame and a position at which the reflection section 1501 is detected in the adjacent second frame. In addition, the reference numeral Δθᵤ₂ represents an angular difference between a position in the circumferential direction at which the reflection section 1501 is detected in the second frame and a position at which the reflection section 1501 is detected in the adjacent third frame. In the following, similarly, the reference numerals Δθᵤ₃, Δθᵤ₄, and Δθᵤ₅ respectively represent angular differences of the positions in the circumferential direction at which the reflection section 1501 is detected between third frame and the fourth frame, between the fourth frame and the fifth frame, and between the fifth frame and the sixth frame.

In addition, the reference numeral Lᵤ₁ represents a position in the axial direction at which the reflection section 1501 is detected in the first frame, based on the position before the ultrasound transceiver 310 starts to move in the axial direction (equal to the distance corresponding to the count value obtained by counting the pulse signals 541 output from the movement amount detector of the linear drive apparatus 407). In addition, the reference numeral Lᵤ₂ represents a position in the axial direction at which the reflection section 601 is detected in the second frame. In the following, similarly, the reference numerals Lᵤ₃, Lᵤ₄, and Lᵤ₅ respectively represent positions in the axial direction at which the reflection section 601 is detected in the third, fourth, and fifth frames.

Similarly, in portion 16b of FIG. 16, the reference numeral Δθₒ₁ represents an angular difference between a position in the circumferential direction at which the reflection section 1501 is detected in the first frame and a position in the circumferential direction at which the reflection section 1501 is detected in the adjacent second frame. In addition, the reference numeral Δθₒ₂ represents an angular difference between a position in the circumferential direction at which the reflection section 1501 is detected in the second frame and a position in the circumferential direction at which the reflection section 1501 is detected in the adjacent third frame. In the following, similarly, the reference numerals Δθₒ₃, Δθₒ₄, and Δθₒ₅ respectively represent angular differences of the positions in the circumferential direction at which the reflection section 1501 is detected between the third frame and the fourth frame, between the fourth frame and the fifth frame, and between the fifth frame and the sixth frame.

In addition, the reference numeral Lₒ₁ represents a position in the axial direction at which the reflection section 1501 is detected in the first frame, based on the position before the ultrasound transceiver 310 starts to move in the axial direction (equal to a value obtained by adding a distance Lz (unknown in the present embodiment) in the axial direction between the ultrasound transceiver 310 and the optical transceiver 320 to the distance corresponding to the count value obtained by counting the pulse signals 541 output from the movement amount detector of the linear drive apparatus 407). As described above, the optical transceiver 320 can be arranged at a position separated to the further proximal end side by the distance Lz (unknown) as compared to the ultrasound transceiver 310. Accordingly, the position of the first frame of the data for ultrasound tomographic image and the position of the first frame of the data for optical tomographic image can be deviated or offset from each other by the distance Lz. In addition, the reference numeral Lᵤ₂ represents the position in the axial direction at which the reflection section 1501 is detected, in the second frame, and the reference numerals Lᵤ₃, Lᵤ₄, and Lᵤ₅ respectively represent the positions in the axial direction at which the reflection section 1501 is detected in the third, fourth, and fifth frames.

As illustrated in portions 16a and 16b of FIG. 16, in a case of the calibration tool 1500, the reflection section 1501 is arranged in a spiral shape, and the pitch of the spiral is gradually widened in the axial direction. Accordingly, the angular difference of the reflection section 1501 between the respective frames is not constant, and gradually decreases as the frame moves forward.

### 11. Calibration Process in Calibration Unit

Next, the calibration process in the calibration unit 506 will be described. Flow in the calibration process in the calibration unit 506 is the same as that in FIG. 10. However, in the graph created in Step S1005, the plotted result and the calculated approximation when the calculated value in Step S1003 and Step S1004 is plotted are different therefrom. Similarly, in the graph created in Step S1015, the plotted result and the calculated approximation when the calculated value in Step S1013 and Step S1014 is plotted are different therefrom.

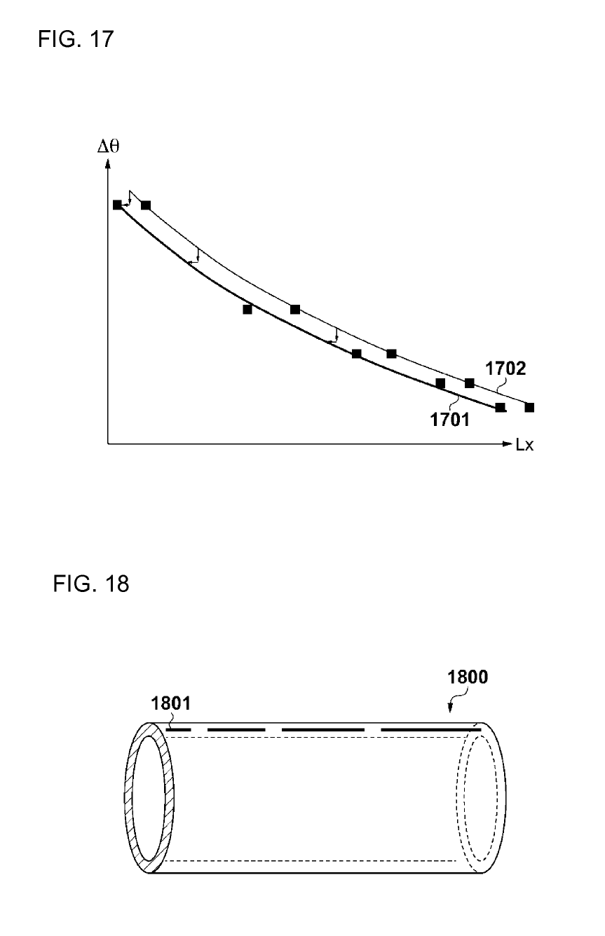

FIG. 17 is a view illustrating the graph and the approximation, which can be created by performing the calibration process using the calibration tool 1500.

In FIG. 17, the reference numeral 1701 illustrates the approximation calculated by plotting (Lᵤ₁, Δθᵤ₁), (Lᵤ₂, Δθᵤ₂), (Lᵤ₃, Δθᵤ₃), (Lᵤ₄, Δθᵤ₄), and (Lᵤ₅, Δθᵤ₅), which are calculated in Step S1003 and Step S1004 with respect to the calibration tool 1500. In addition, the reference numeral 1702 illustrates the approximations calculated by plotting (Lₒ₁, Δθₒ₁), (Lₒ₂, Δθₒ₂), (Lₒ₃, Δθₒ₃), (Lₒ₄, Δθₒ₄), and (Lₒ₅, Δθₒ₅) which are calculated in Step S1013 and Step S1014 with respect to the calibration tool 1500.

As illustrated in FIG. 17, when the calibration process is performed by using the calibration tool 1500 in which the reflection section 1501 is arranged in a spiral shape and is arranged so that the pitch of the spiral is gradually narrowed along the axial direction, the approximations 1701 and 1702 have the same shape, but are deviated to the horizontal axis and the vertical axis.

In accordance with an exemplary embodiment, for example, the approximation 1701 is deviated in the horizontal axis direction and the vertical axis direction, and thus can be superimposed on the approximation 1702. At this time, a deviated amount in the horizontal axis direction is equal to the distance difference in the axial direction between the transmitting and receiving direction of the ultrasound transceiver 310 and the transmitting and receiving direction of the optical transceiver 320. In addition, a deviated amount in the vertical axis direction is equal to the angular difference in the circumferential direction between the transmitting and receiving direction of the ultrasound transceiver 310 and the transmitting and receiving direction of the optical transceiver 320.

In accordance with an exemplary embodiment, by calculating the deviated amounts for superimposing the approximation 1701 and the approximation 1702 on each other, the distance difference in the axial direction and the angular difference in the circumferential direction between the transmitting and receiving direction of the ultrasound transceiver 310 and the transmitting and receiving direction of the optical transceiver 320 can be obtained.

As is apparent from the above description, the present embodiment adopts a configuration in which the distance difference in the axial direction and the angular difference in the circumferential direction between the ultrasound transceiver and the optical transceiver can be calculated by using the calibration tool which is formed in the hollow cylindrical shape and in which the reflection section is arranged in the spiral shape and is arranged so that the pitch of the spiral is gradually narrowed along the axial direction.

As a result, even when the distance difference in the axial direction and the angular difference in the circumferential direction between the ultrasound transceiver and the optical transceiver are not known, the position correction process can be performed according to the distance difference and the angular difference by performing the calibration process using the calibration tool.

In the above-described first exemplary embodiment, a case has been described where the reflection section of the calibration tool is configured to have the continuous straight line. However, the present disclosure is not limited thereto. For example, the reflection section of the calibration tool may be configured to have a discontinuous straight line (dashed line). Hereinafter, details of the present embodiment will be described.
(1) Description of Calibration Tool

FIG. 18 is a view illustrating an example of a calibration tool 1800 according to the present embodiment. As illustrated in FIG. 18, the calibration tool 1800 has a hollow cylindrical shape, and can be configured so that the imaging core 220 is inserted into the calibration tool 1800. A linear reflection section 1801 arranged substantially parallel to the axial direction is arranged on an inner wall surface or an outer wall surface of the calibration tool 1800. The reflection section 1801 can be configured to have a dashed line which is discontinuous in the axial direction and in which a lined portion and a lineless portion are alternately repeated.

However, the reflection section 1801 can be configured so that whereas the lengths of the lineless portions are respectively constant, the lengths of the lined portions are gradually lengthened as the lined portions move forward in the axial direction.
(2) Example of Data for Ultrasound Tomographic Image and Data for Optical Tomographic Image

Next, an example of the data for ultrasound tomographic image and the data for optical tomographic image which are acquired when the calibration process is performed by using the calibration tool 1800 will be described.

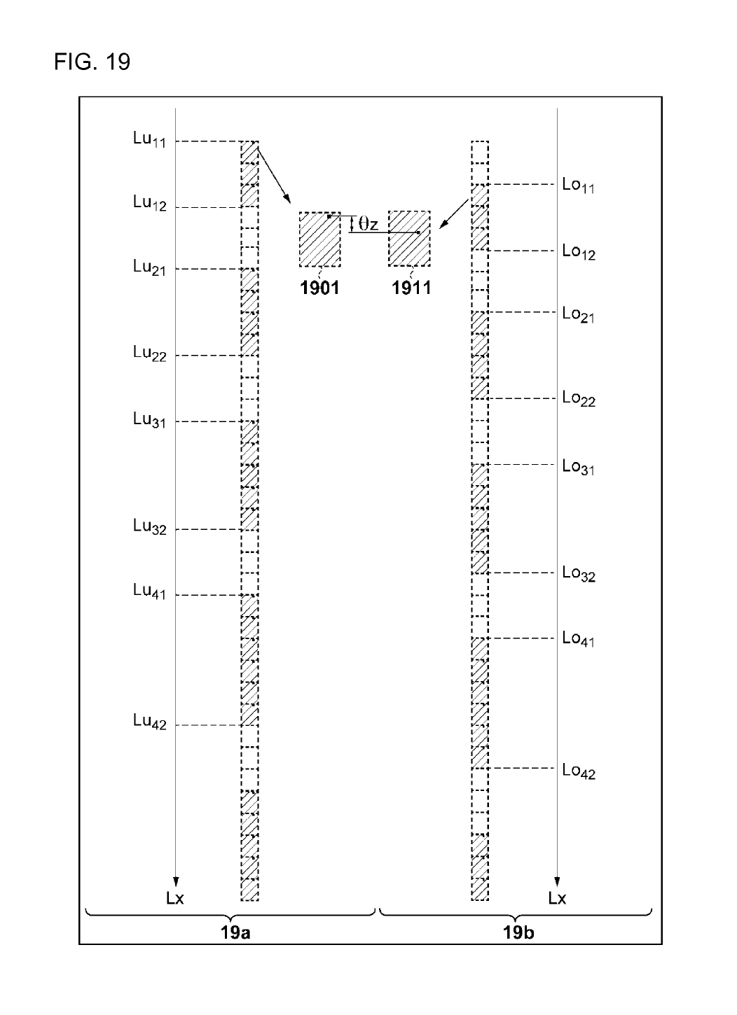

FIG. 19 is a diagram illustrated by hatching a frame from which the reflection section 1201 is detected among the respective frames of the data for ultrasound tomographic image and a frame from which the reflection section 1801 is detected among the respective frames of the data for optical tomographic image.

In portion 19a of FIG. 19, the reference numeral Lᵤ₁₁ represents a position in the axial direction of a frame from which the first lined portion of the reflection section 1801 is detected for the first time within the data for ultrasound tomographic image. In addition, the reference numeral Lᵤ₁₂ represents a position in the axial direction of a frame from which the first lined portion of the reflection section 1801 is detected for the last time. In addition, the reference numeral Lᵤ₂₁ represents a position in the axial direction of a frame from which the second lined portion of the reflection section 1801 is detected for the first time. In addition, the reference numeral Lᵤ₂₂ represents a position in the axial direction of a frame from which the second lined portion of the reflection section 1801 is detected for the last time. In the following, similarly, the reference numerals Lᵤ₃₁, Lᵤ₃₂, Lᵤ₄₁, and Lᵤ₄₂ respectively represent positions in the axial direction of the frames from which the third and fourth lined portions of the reflection section 1801 are detected for the first time or positions in the axial direction of the frames from which the third and fourth lined portions of the reflection section 1801 are detected for the last time.

Similarly, in portions 19b of FIG. 19, the reference numeral Lₒ₁₁ represents a position in the axial direction of a frame from which the first lined portion of the reflection section 1801 is detected for the first time within the data for optical tomographic image. In addition, the reference numeral Lₒ₁₂ represents a position in the axial direction of a frame from which the first lined portion of the reflection section 1801 is detected for the last time. In addition, the reference numeral Lₒ₂₁ represents a position in the axial direction of a frame from which the second lined portion of the reflection section 1801 is detected for the first time. In addition, the reference numeral Lₒ₂₂ represents a position in the axial direction of a frame from which the second lined portion of the reflection section 1801 is detected for the last time. In the following, similarly, the reference numerals Lₒ₃₁, Lₒ₃₂, Lₒ₄₁, and Lₒ₄₂ respectively represent positions in the axial direction of the frames from which the third and fourth lined portions of the reflection section 1801 can be detected for the first time or positions in the axial direction of the frames from which the third and fourth lined portions of the reflection section 1801 can be detected for the last time.

As illustrated in portions 19a and 19b of FIG. 19, in a case of the calibration tool 1800, the length of the lined portion of the reflection section 1801 is gradually lengthened. Therefore, the data for ultrasound tomographic image or the data for optical tomographic image can be deviated or offset in the axial direction. In this manner, the length (for example, Lᵤ₁₂-Lᵤ₁₁) of the lined portion in the data for ultrasound tomographic image and the length (for example, Lₒ₁₂-Lₒ₁₁) of the lined portion in the data for optical tomographic image can coincide with each other (position for enabling the coincidence is uniquely determined).

The deviated amount at this time is equal to the distance difference in the axial direction between the transmitting and receiving direction of the ultrasound transceiver 310 and the transmitting and receiving direction of the optical transceiver 320. For example, the deviated amount when the data for ultrasound tomographic image or the data for optical tomographic image is deviated in the axial direction is obtained so that the length of the lined portion in the data for ultrasound tomographic image and the length of the lined portion in the data for optical tomographic image coincide with each other. In this manner, the distance difference can be calculated in the axial direction between the transmitting and receiving direction of the ultrasound transceiver 310 and the transmitting and receiving direction of the optical transceiver 320.

In a state where the data for ultrasound tomographic image or the data for optical tomographic image is deviated in the axial direction and the length of the lined portion in the data for ultrasound tomographic image and the length of the lined portion in the data for optical tomographic image can coincide with each other, detection positions in the circumferential direction of the reflection section 1801 inside the corresponding frame can be compared with each other. In this manner, the angular difference can be obtained in the circumferential direction between the transmitting and receiving direction of the ultrasound transceiver 310 and the transmitting and receiving direction of the optical transceiver 320.

Description continues with reference to FIG. 19. In FIG. 19, the reference numeral 1901 is a frame from which the first lined portion is detected for the first time within the data for ultrasound tomographic image, and the reference numeral 1911 is a frame from which the first lined portion is detected for the first time within the data for optical tomographic image. The frame 1901 and the frame 1911 are frames corresponding to each other in a state where the data for ultrasound tomographic image or the data for optical tomographic image is deviated in the axial direction and the length of the lined portion in the data for ultrasound tomographic image and the length of the lined portion in the data for optical tomographic image are caused to coincide with each other.

Here, an angular difference θz between the position in the circumferential direction at which the lined portion of the reflection section 1801 is detected in the frame 1901 and the position in the circumferential direction at which the lined portion of the reflection section 1801 is detected in the frame 1911 is equal to the angular difference in the circumferential direction between the transmitting and receiving direction of the ultrasound transceiver 310 and the transmitting and receiving direction of the optical transceiver 320.

In accordance with an exemplary embodiment, the angular difference in the circumferential direction can be calculated between the transmitting and receiving direction of the ultrasound transceiver 310 and the transmitting and receiving direction of the optical transceiver 320 by obtaining the angular difference Oz between the position in the circumferential direction at which the lined portion of the reflection section 1801 is detected in each frame of the ultrasound tomographic image and the position in the circumferential direction at which the lined portion of the reflection section 1801 is detected in each frame of the optical tomographic image.

As is apparent from the above description, the present embodiment adopts a configuration in which the distance difference in the axial direction and the angular difference in the circumferential direction between the ultrasound transceiver and the optical transceiver can be calculated by using the calibration tool which is formed in the hollow cylindrical shape and in which the discontinuous and linear reflection section is arranged substantially parallel to the axial direction.

As a result, even when the distance difference in the axial direction and the angular difference in the circumferential direction between the ultrasound transceiver and the optical transceiver are not known, the position correction process can be performed according to the distance difference and the angular difference by performing the calibration process using the calibration tool.

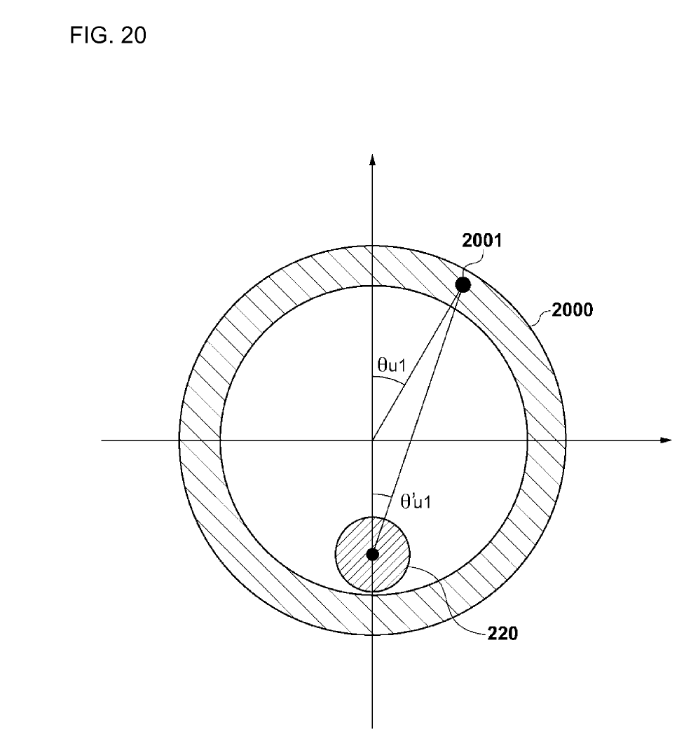

In the above-described first to fourth embodiments, a case has been described on the assumption that the imaging core 220 is rotatably operated at the center position of the calibration tool. However, the present invention is not limited thereto. For example, as illustrated in FIG. 20, when an inner diameter of a calibration tool 2000 is larger than a cross-sectional area of the imaging core 220, it is also considered that the imaging core 220 is rotatably operated at a position deviated from the center position of the calibration tool 2000.

In this case, an angle from a frame end of a reflection section 2001 detected in each frame is originally to be calculated as θᵤ₁. However, it is actually calculated as θ′ᵤ₁. For this reason, in order to perform the calibration process, it can be desirable to perform a process of converting the angle into the angle θᵤ₁ calculated when the imaging core 220 is rotatably operated at the center position of the calibration tool 2000.

In addition, in the above-described first to fourth embodiments, a case has been described on the assumption that the ultrasound transceiver and the optical transceiver are arranged in the imaging core 220. However, the present disclosure is not limited thereto. The same calibration process can also be applied to a case where two ultrasound transceivers are arranged in the imaging core 220, or a case where two optical transceivers are arranged therein. In addition, the number of transceivers arranged in the imaging core 220 is not limited to two, and may be three or more. The calibration process described in the above-described first to fourth embodiments can also be applied to this case.

In addition, the above-described first to fourth embodiments adopt a configuration in which the position correction is performed on the ultrasound tomographic image based on the correction value calculated as a result of the calibration process. However, without being limited thereto, the present disclosure may be configured so that the position correction is performed on the optical tomographic image. Alternatively, a configuration may be adopted so that the position correction is performed on both the ultrasound tomographic image and the optical tomographic image.

In addition, the above-described fourth embodiment adopts a configuration in which the length of the lineless portion is constant and the length of the lined portion is gradually lengthened along the axial direction in order to configure the reflection section 1801. However, without being limited thereto, the present disclosure may adopt a configuration in which the length of the lined portion is fixed and the length of the lineless portion is gradually lengthened along the axial direction.

The detailed description above describes an imaging apparatus for diagnosis, a calibration tool for calibrating the imaging apparatus for diagnosis, and a calibration method of the imaging apparatus for diagnosis using the calibration tool. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, the apparatus comprising:
generation means for generating the first tomographic image of a calibration tool based on the first signal transmitted and received by the first transceiver, and for generating the second tomographic image of the calibration tool based on the second signal transmitted and received by the second transceiver, the calibration tool having a reflection section arranged to reflect the first signal and the second signal and a lumen into which the transceiver is inserted;
calculation means for calculating an angular difference in a circumferential direction around an axis between the first transceiver and the second transceiver, based on positional information of the reflection section which is detected on the first tomographic image of the calibration tool and positional information of the reflection section which is detected on the second tomographic image of the calibration tool; and
correction means for correcting an angle in the circumferential direction of the first tomographic image or the second tomographic image inside the lumen of the measurement object body according to the angular difference calculated by the calculation means, when displaying the first tomographic image and the second tomographic image inside the lumen of the measurement object body.

2. The imaging apparatus for diagnosis according to claim 1, wherein the reflection section is arranged in a spiral shape at a constant pitch in the axial direction in the calibration tool, and
the calculation means calculates an angular difference in the circumferential direction by calculating a difference between an angle from a frame end of the reflection section detected in each frame on the first tomographic image of the calibration tool and an angle from a frame end of the reflection section detected in each frame on the second tomographic image of the calibration tool.

3. The imaging apparatus for diagnosis according to claim 1, wherein
the reflection section is arranged in a spiral shape in the calibration tool, and is arranged so that a pitch of the spiral is changed in the axial direction of the calibration tool, and
the calculation means calculates an angular difference in the circumferential direction and a distance difference in the axial direction between the first transceiver and the second transceiver by using a first approximation calculated based on an angular difference in the circumferential direction between the adjacent frames of the reflection section detected in each frame on the first tomographic image of the calibration tool and a position in the axial direction of the reflection section detected in each frame and a second approximation calculated based on an angular difference in the circumferential direction between the adjacent frames of the reflection section detected in each frame on the second tomographic image of the calibration tool and a position in the axial direction of the reflection section detected in each frame.

4. The imaging apparatus for diagnosis according to claim 3, wherein
the calculation means calculates an angular difference in the circumferential direction and a distance difference in the axial direction between the first transceiver and the second transceiver, based on a movement amount when the first approximation or the second approximation is moved so that the first approximation and the second approximation are superimposed on each other.

5. The imaging apparatus for diagnosis according to claim 1, wherein
the reflection section is formed to have a straight line which is substantially parallel to the axial direction of the calibration tool, and
the calculation means calculates an angular difference in the circumferential direction by calculating a difference between an angle from a frame end of the reflection section detected in each frame on the first tomographic image of the calibration tool and an angle from a frame end of the reflection section detected in each frame on the second tomographic image of the calibration tool.

6. The imaging apparatus for diagnosis according to claim 5, wherein
the reflection section is formed to have a dashed line in which a lined portion and a lineless portion are alternately repeated, and either the length of the lined portion or the length of the lineless portion of the dashed line is changed in the axial direction of the calibration tool, and
the calculation means calculates a distance difference in the axial direction between the first transceiver and the second transceiver by calculating a movement amount when the first tomographic image of the calibration tool or the second tomographic image of the calibration tool is moved in the axial direction so that a continuous length of a frame from which the reflection section is detected on the first tomographic image of the calibration tool and a continuous length of a frame from which the reflection section is detected on the second tomographic image of the calibration tool coincide with each other.

7. The imaging apparatus for diagnosis according to claim 1, wherein
positional information of the reflection section includes an angle in the circumferential direction around an axis, and
the angle in the circumferential direction around the axis is an angle converted into an angle obtained when the first and second transceivers move a center position of the calibration tool in the axial direction.

8. A calibration method of an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, the method comprising:
a generation step of generating the first tomographic image of a calibration tool based on the first signal transmitted and received by the first transceiver and generating the second tomographic image of the calibration tool based on the second signal transmitted and received by the second transceiver, with regard to the calibration tool which has a reflection section arranged to reflect the first signal and the second signal and has a lumen into which the transceiver is inserted;
a calculation step of calculating an angular difference in a circumferential direction around an axis between the first transceiver and the second transceiver, based on positional information of the reflection section which is detected on the first tomographic image of the calibration tool and positional information of the reflection section which is detected on the second tomographic image of the calibration tool; and
a correction step of correcting an angle in the circumferential direction of the first tomographic image or the second tomographic image inside the lumen of the measurement object body according to the angular difference calculated by the calculation step, when displaying the first tomographic image and the second tomographic image inside the lumen of the measurement object body.

9. The calibration method according to claim 8, comprising:
arranging the reflection section in a spiral shape at a constant pitch in the axial direction in the calibration tool, and
the calculation step calculates an angular difference in the circumferential direction by calculating a difference between an angle from a frame end of the reflection section detected in each frame on the first tomographic image of the calibration tool and an angle from a frame end of the reflection section detected in each frame on the second tomographic image of the calibration tool.

10. The calibration method according to claim 8, wherein
arranging the reflection section in a spiral shape in the calibration tool, and is arranged so that a pitch of the spiral is changed in the axial direction of the calibration tool, and
the calculation step calculates an angular difference in the circumferential direction and a distance difference in the axial direction between the first transceiver and the second transceiver by using a first approximation calculated based on an angular difference in the circumferential direction between the adjacent frames of the reflection section detected in each frame on the first tomographic image of the calibration tool and a position in the axial direction of the reflection section detected in each frame and a second approximation calculated based on an angular difference in the circumferential direction between the adjacent frames of the reflection section detected in each frame on the second tomographic image of the calibration tool and a position in the axial direction of the reflection section detected in each frame.

11. The calibration method according to claim 10, wherein
the calculation step calculates an angular difference in the circumferential direction and a distance difference in the axial direction between the first transceiver and the second transceiver, based on a movement amount when the first approximation or the second approximation is moved so that the first approximation and the second approximation are superimposed on each other.

12. The calibration method according to claim 8, wherein
forming the reflection section to have a straight line which is substantially parallel to the axial direction of the calibration tool, and
the calculation step calculates an angular difference in the circumferential direction by calculating a difference between an angle from a frame end of the reflection section detected in each frame on the first tomographic image of the calibration tool and an angle from a frame end of the reflection section detected in each frame on the second tomographic image of the calibration tool.

13. The calibration method according to claim 12, wherein
forming the reflection section to have a dashed line in which a lined portion and a lineless portion are alternately repeated, and either the length of the lined portion or the length of the lineless portion of the dashed line is changed in the axial direction of the calibration tool, and
the calculation step calculates a distance difference in the axial direction between the first transceiver and the second transceiver by calculating a movement amount when the first tomographic image of the calibration tool or the second tomographic image of the calibration tool is moved in the axial direction so that a continuous length of a frame from which the reflection section is detected on the first tomographic image of the calibration tool and a continuous length of a frame from which the reflection section is detected on the second tomographic image of the calibration tool coincide with each other.

14. The calibration method according to claim 8, wherein
positional information of the reflection section includes an angle in the circumferential direction around an axis, and
the angle in the circumferential direction around the axis is an angle converted into an angle obtained when the first and second transceivers move a center position of the calibration tool in the axial direction.

15. A non-transitory computer-readable recording medium with a program stored therein which causes a computer to execute each process of the calibration method according to claim 8.

16. A calibration tool for calibrating an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, comprising:
a reflection section to reflect the first signal and the second signal, wherein the reflection section is arranged in a spiral shape along the axial direction; and
a lumen into which the transceiver is inserted.

17. A calibration tool for calibrating an imaging apparatus for diagnosis in which when a transceiver having a first transceiver configured to transmit and receive a first signal and a second transceiver configured to transmit and receive a second signal moves in an axial direction while rotating inside a lumen of a measurement object body, a first tomographic image and a second tomographic image inside the lumen of the measurement object body are generated by using the first signal transmitted and received by the first transceiver and the second signal transmitted and received by the second transceiver, comprising:
a reflection section to reflect the first signal and the second signal, wherein the reflection section is formed to have a straight line which is substantially parallel to the axial direction is arranged; and
a lumen into which the transceiver is inserted.
